(19) United States
(12) Patent Application Publication
(10) Pub. No.: US 2025/0276107 A1
(43) Pub. Date: Sep. 4, 2025

(54) HYDROGEL SYSTEMS AND METHODS FOR TREATING FISTULA AND OTHER INDICATIONS

(71) Applicants: Terasaki Institute for Biomedical Innovation, Los Angeles, CA (US); Rousselot B.V., Gent (BE)

(72) Inventors: Reihaneh Haghniaz, Los Angeles, CA (US); Alireza Khademhosseini, Los Angeles, CA (US); Fatemeh Zehtabi, Winnetka, CA (US); Hossein Montazerianoliaee, Boston, MA (US); Fahimeh Zarei, Reseda, CA (US); Bjorn Vergauwen, Gent (BE)

(73) Assignees: Terasaki Institute for Biomedical Innovation, Los Angeles, CA (US); Rousselot B.V., Gent (BE)

(21) Appl. No.: 19/068,219

(22) Filed: Mar. 3, 2025

Related U.S. Application Data
(60) Provisional application No. 63/561,146, filed on Mar. 4, 2024.

(51) Int. Cl. A61L 24/00; A61L 24/04

(52) U.S. Cl. CPC A61L 24/0031; A61L 24/0015; A61L 24/046; A61L 2300/41; A61L 2300/414

(57) ABSTRACT

The present disclosure generally relates, in certain aspects, to hydrogel systems, for example, for treating fistulas and other indications. In some embodiments, the hydrogel system comprises a cross-linked polymer network comprising an adhesion promoter, a thiolated-polymer, and a multifunctional cross-linker. The hydrogel system may be configured to adhere to one or more substrates, for example, a biological tissue. In some embodiments, the adhesion is enhanced and/or mediated by the adhesion promoter, for example, polydopamine. In certain embodiments, the hydrogel system is configured to form an adhesive plug within a cavity, e.g., a biological cavity such as a fistula. The hydrogel system may further encapsulate one or more drugs, including but not limited to antibiotic, and/or antifungal agents, and/or wound healing accelerators, configured to release from the hydrogel system over a period of time. Other embodiments are generally directed toward methods of administering the hydrogels systems to a subject, devices involving these, or the like.

### RELATED APPLICATIONS

This application claims the benefit of U.S. Provisional Patent Application Ser. No. 63/561,146, filed Mar. 4, 2024, entitled “Hydrogel Systems and Methods for Treating Fistula and Other Indications,” which is incorporated herein by reference in its entirety.

### TECHNICAL FIELD

The present disclosure generally pertains to articles and methods for the creation of crosslinked materials for a myriad of applications. These applications include but are not limited to bioadhesives and/or sealants for abnormal cavities (such as fistulas, aneurysms, tooth/gum cavities, tumor resection void), tissue augmentation, ulcers and sores, coating surfaces of implants, vascular malformation (e.g., arteriovenous/venous malformation), drug delivery matrices, and/or hemostats, among others. Other applications of hydrogel systems are also described.

### BACKGROUND

Abnormal cavities, such as fistulas and aneurysms, present significant challenges in medical treatment due to their association with high morbidity, mortality, and compromised quality of life. Traditional treatment modalities, including surgery and non-operative interventions like fillers, hydrogels, plugs, glues, and sealants, often face limitations in terms of adhesive strength, mechanical stability, and biological efficacy. Surgical approaches, while effective, carry risks and may not be suitable for all patients.

Current bioadhesives utilized in these applications exhibit shortcomings, primarily in their inability to provide adequate adhesion and mechanical stability during in situ injection. Moreover, they often lack essential biological effects, such as antibacterial or wound-healing properties, crucial for addressing the complex nature of abnormal cavities, particularly those associated with chronic diseases.

There exists a need for a revolutionary approach to address these challenges, offering a biomaterial that not only adheres effectively to the abnormal cavity but also provides mechanical stability and imparts therapeutic benefits for enhanced healing and patient well-being.

### SUMMARY

Articles and methods using hydrogel systems to seal body cavities are generally described. Other applications of hydrogel systems are also described. The subject matter of the present disclosure involves, in some cases, interrelated products, alternative solutions to a particular problem, and/or a plurality of different uses of one or more systems and/or articles. Aspects of the present disclosure relate to articles comprising polydopamine, a maleimide-functionalized cross-linker, and a thiolated-polymer. In some embodiments, the articles comprises an adhesion promoter selected from the group consisting of, indole-5,6-quinone, eumelanin, melanin, pheomelanin, 5,6-dihydroxyindole, tyrosine, L-dopaquinone, L-leucodopachrome, serotonin, and L-dopachrome, caffeic acid, polycaffeic acid and tanic acid; a multi-functional crosslinker; and a thiolated-polymer.

Other aspects of the present disclosure relate to methods, for example, for forming a cross-linked polymer network. In some embodiments, the method comprises forming a cross-linked polymer network by combining a first fluid and a second fluid, the first fluid comprising polydopamine and a maleimide-functionalized crosslinker, and the second fluid comprising a thiolated-polymer. In some embodiments, the method comprises administering a mixture into a body cavity in a subject, wherein the mixture comprises a first fluid and a second fluid, the first fluid comprising polydopamine and a maleimide-functionalized crosslinker, and the second fluid comprising a thiolated-polymer.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 depicts a fabrication process of adhesive hydrogel and its facile application for fistula treatment, according to some embodiments.

FIG. 2A depicts structural changes of DA during oxidative polymerization, according to some embodiments.

FIG. 2B depicts (I) time-dependent UV-Vis absorption spectra of DA in Tris-HCl buffer (pH 8.5) and photographs of the samples at each time point. UV-Vis absorption peaks of DA (II) at 280 nm, and (III) 420 nm over time, according to some embodiments.

FIG. 2C depicts (I) transmission electron microscope images of PDA after developing in Tris-HCl buffer. (II) hydrodynamic size of PDA at neutral pH versus alkaline pH. (III) zeta-potential of PDA at pH=7 and pH=9.5.

FIG. 2D depicts (I) ¹HNMR and (II) FTIR spectra, demonstrating signature peaks for DA and PDA at various pH, according to some embodiments.

FIG. 3A depicts chemical synthesis of a hydrogel, according to some embodiments.

FIG. 3B depicts (I) FTIR, and (II) NMR spectra of GS-PEM-PDA crosslinked hydrogel versus its precursors, according to some embodiments.

FIG. 3C depicts (I) the effect of initial pH of PDA on gel formation time of GS10%-PEM10%-PDA1.25%, (II) the effect of GS and PDA (at pH=9.5) concentrations on dynamic moduli of hydrogels. (III) the gel formation times of different hydrogel formulations (IV) the storage modulus of hydrogels at gel formation time, and (V) the storage modulus of hydrogels ˜15 min after gelation (the dashed lines indicate the resistant threshold for occlusion at 300 mmHg), according to some embodiments.

FIG. 4A depicts (I) a schematic of the injection force measurement setup. (II) the results of injection force for GS 20% wt/v, and (III) GS 30% wt/v, injected using a 1 ml syringe attached to the 4F catheter (length of 11 cm) at 33.33 mm/min, according to some embodiments.

FIG. 4B depicts (I) a schematic of the compression study setup. (II) Representative compressive stress (kPa) versus strain (mm/mm) curve for hydrogels at varying concentrations of alkaline PDA (0, 1.25 and 2.5% wt/v). (III) Quantitative compressive strength (kPa) for different hydrogels, according to some embodiments.

FIG. 4C depicts (I) the swelling ratio of different hydrogels at varying concentrations of alkaline PDA up to 48 h. (II) Degradation profile of hydrogels in terms of mass remained (%) in response to incubation time in collagenase (2.5 U/ml) at 37° C. (III) Cumulative PDA release profile (mg) per ml of gel for up to 340 h, according to some embodiments.

FIG. 5A depicts (I) an schematic illustration of wound closure test and the adhesive strength (kPa) for different hydrogels versus commercial glue, Tisseel. (II) An schematic illustration of lap shear test and shear strength (kPa) of hydrogels and Tisseel evaluated from lap shear test, according to some embodiments.

FIG. 5B depicts (I) an schematic illustration of the fistula ex vivo bench test. (II) Pressure of the occluded fistula model using different hydrogels or Tisseel, at the flow rate of 30 ml/min, and (II) flow rate of 120 ml/min. The dashed lines show the minimum pressure to assess the success rate of the hydrogels. (III) Success rate of fistula tract occlusion by different hydrogels at flow rate of 120 ml/min, according to some embodiments.

FIG. 6A depicts (I) gross images of in vitro hemolysis assay using diluted human blood exposed to different hydrogels versus Triton-X-100 (positive control) and saline (negative control). (II) Quantified hemolysis percentage for various hydrogels, according to some embodiments.

FIG. 6B depicts (I) live/dead fluorescent images of human dermal fibroblast cells (HDF) cultured for 5 days with hydrogel elutes versus untreated cells served as control. (II) HDF cell viability up to day 5 of incubation with different hydrogels. (III) Metabolic activity of the HDF cells estimated using PrestoBlue assay after 1, 3, and 5 days incubation with hydrogel elutes, according to some embodiments. Data are displayed as mean values of 4 replicates +/−standard deviation. The significant differences were analyzed by two-way ANOVA with multiple comparisons test. Asterisks indicate statistically significant results with p-values <0.0001 (****). The ‘ns’ indicates no significant differences.

FIG. 7A depicts (I) a photograph of inhibition zone after challenging E. coli for 12 h with hydrogels or ciprofloxacin (8 mg/ml), a broad-spectrum antibiotic as a positive control. (II) Quantified area of bacterial zone of inhibition after subjecting E. coli to hydrogels for 12 h, according to some embodiments.

FIG. 7B depicts (I) a representative images of migration assay after incubation of HDF with hydrogel elutes for 0, 2, 6 and 12 h. Scale bara show 500 micrometers). (II) A quantitative analysis of cell migration assay demonstrating the effect of bioadhesives on artificial wound closure, versus epidermal growth factor (EPG, served as a positive control) and untreated wound (served as a negative control), according to some embodiments. Data are displayed as mean values of 3 replicates +/−standard deviation. The significant differences were analyzed by two-way ANOVA with multiple comparisons test. Asterisks indicate statistically significant results with p-values <0.05 (*), <0.01 (**), <0.001 (***), or <0.0001 (****). The ‘ns’ indicates no significant differences.

FIG. 8A depicts storage moduli (G′) of hydrogels containing anti-inflammatory drug (BPC-157, 0.05% v/v) and/or Omnipaque contrast agent (CA, 25% v/v) 15 min after gel formation, according to some embodiments.

FIG. 8B depicts the success rate of various hydrogels containing Omnipaque contrast agent (CA, 25% v/v) to occlude in vitro phantom (diameter=3 mm, length=1 cm), mimicking fistula model, according to some embodiments.

FIG. 8C depicts a radiopacity evaluation of commercial contrast agent (CA), Omnipaque 300 mg/ml, under fluoroscopy for (I) CA100%, (II) GS 20%-CA50%, (III) GS10%-PDA2.5%-CA25% and (IV) GS10%-PDA1.25%-CA 25%, according to some embodiments.

FIG. 8D depicts a radiopacity evaluation of commercial contrast agent (CA), Omnipaque 300 mg/ml, under computed tomography (CT) scan for (I) CA100%, (II) GS 20%-CA50%, (III) GS10%-PDA2.5%-CA25% and (IV) GS10%-PDA1.25%-CA 25%, according to some embodiments.

FIG. 8E depicts metabolic activity of HDF cells after incubation with different concentrations of BPC-157 (ranging from 0 to 500 micrograms/ml) for 1 and 4 days, using PrestoBlue assay, according to some embodiments.

FIG. 8F depicts gross image of inhibition zone after challenging E. coli for 12 h with different concentrations of BPC-157 versus ciprofloxacin (8 mg/ml), a broad-spectrum antibiotic as a positive control, according to some embodiments.

FIG. 8G depicts area of bacterial inhibition zone after subjecting E. coli with different concentrations of free-BPC for 24 h, according to some embodiments.

### DETAILED DESCRIPTION

The present disclosure generally relates, in certain aspects, to hydrogel systems, for example, for treating fistulas and other indications. In some embodiments, the hydrogel system comprises a cross-linked polymer network comprising an adhesion promoter, a thiolated-polymer, and a multifunctional cross-linker. The hydrogel system may be configured to adhere to one or more substrates, for example, a biological tissue. In some embodiments, the adhesion is enhanced and/or mediated by the adhesion promoter, for example, polydopamine. In certain embodiments, the hydrogel system is configured to form an adhesive plug within a cavity, e.g., a biological cavity such as a fistula. The hydrogel system may further encapsulate one or more pharmaceutical agents, including but not limited to anti-inflammatory agents, wound-healing agents, antibiotic and/or antifungal agents, configured to release from the hydrogel system over a period of time. Other embodiments are generally directed toward methods of administering the hydrogels systems to a subject, devices involving these, or the like.

Certain aspects of the disclosure relate to forming the cross-linked polymer network by combing a first fluid and a second fluid. In some embodiments, the first fluid comprises the adhesion promoter and the multi-functional crosslinker. In some embodiments, the second fluid comprises a thiolated-polymer. The cross-linked polymer network may be formed upon physical contact between the first fluid and the second fluid. Accordingly, the first and second fluid may be combined to form the cross-linked polymer network.

The first fluid generally described herein may have any of variety of suitable pH's. In some embodiments, the pH of the first fluid is greater than or equal to 7, greater than or equal to 7.5, greater than or equal to 8, greater than or equal to 8.5, greater than or equal to 9, greater than or equal to 9.5, greater than or equal to 10, greater than or equal to 10.5, or greater than or equal to 11. In some embodiments, the pH of the first fluid is less than or equal to 11, less than or equal to 10.5, less than or equal to 10, less than or equal to 9.5, less than or equal to 9, less than or equal to 8.5, less than or equal to 8, less than or equal to 7.5, or less than or equal to 7. Combinations of these ranges are possible (e.g., greater than or equal to 7 and less than or equal to 11). Other ranges are possible.

Some aspects of the present disclosure relate to methods of administering the hydrogel systems to a subject in need (e.g., for treatment of a fistula). In some embodiments, the methods relate to administering a mixture comprising a first fluid and a second fluid to a body cavity (e.g., fistula, blood vessel, shunts, etc.) of a subject in need. In some embodiments, administering the mixture within the body cavity, as contemplated herein, produces a plug that completely, or partially, seals a first side of the body cavity from a second side of the body cavity. In certain embodiments, the plug provides a scaffold for cell ingrowth.

Conventional methodologies to treat and/or seal body cavities generally involve surgical operations which often result in post-operative complications. While non-surgical treatments, such as the application of surgical glue, are possible, weak adhesion and the potential risk of inflammation render these treatments are suboptimal as they can dislodge and/or migrate over time. Therefore, certain embodiments are directed to treatments for the sealing and healing of body cavities in subjects.

Without wishing to be bound by any particular theory, it is generally believed that the formation of a plug within the interior cavity of a body cavity, such as a fistula, may serve as a scaffold for cell ingrowth and the formation of a cellular plug. For example, hydrogels comprising gelatin support cell attachment, migration and proliferation may be readily degraded in vivo via a combination of hydrolysis and enzymes (e.g., matrix metalloproteinases, MMPs, carboxypeptidases, aminopeptidases, and dipeptidases, among others). Thus, it is believed that cells within the interior body cavity may infiltrate the adhesive plug and deposit extracellular matrix, and in some cases, while simultaneously degrading the plug to form a native cell-plug within the body cavity.

The present disclosure generally describes a hydrogel system that can advantageously adhere to the body cavity with sufficient strength to prevent dislodgement when under pressure and shear. In some embodiments, the cross-linked polymer network comprises antibacterial, antimicrobial, and/or antifungal properties that promote healing of the body cavity. According to some cases, the cross-linked polymer network and the relative status of the cross-linked polymer network can be tracked via medical imaging techniques to ensure adequate placement, function, and/or degradation. Additionally, the cross-linked polymer network may form in a relatively short period of time, which is highly desirable for relatively quick and precise administration of the hydrogel system. The cross-linked polymer network may advantageously comprise polydopamine (PDA) which, without being bound by any particular theory, allows the cross-linked polymer network to exhibit some of the aforementioned properties favorable for the treatment and sealing of body cavities.

In some embodiments, the cross-linked polymer network comprises an adhesion promoter. Without wishing to be bound by any particular theory, the adhesion promoter comprises chemical moieties that can interact with substrates (e.g., biological tissue) to enhance and/or promote the adhesion of the cross-linked polymer network to the substrate. To facilitate the sealing and/or closure of a body cavity, such as a fistula, the cross-linked polymer network may have sufficient adhesion, mediated by the adhesion promoter, to biological tissues exposed within and/or on the body cavity to mitigate the potential of dislodgement. In some embodiments, the adhesion promoter is configured to covalently interact with polymers and/or copolymers to form the cross-linked network. Accordingly, the adhesion promoter may in certain embodiments advantageously promote crosslinking between the multi-functional maleimide and the thiolated-polymer contributing, in part or in whole, to the mechanical properties of the cross-linked polymer network. The adhesion promoter may also advantageously accelerate the formation of the cross-linked network allowing for relatively quick sealing, closure, and/or occlusion of body cavity upon administering of the first and second fluids.

In certain embodiments, the adhesion promoter comprises polydopamine (PDA). Without wishing to be bound by any particular theory, polydopamine, as opposed to its non-polymeric form (dopamine), can promote adhesion to certain substrates (e.g., biological tissue) through the presence of hydroxyl and/or carbonyl groups on the catechol and quinone moieties, as shown in Example 1. The quinone form of polydopamine, in certain embodiments, can have an increased propensity to covalently interact with thiolated-polymer and/or multi-functional crosslinkers allowing for the formation of the cross-linked polymer network and further promote the adhesion properties of the cross-linked polymer network, e.g., as shown in Example 1. PDA may also advantageously exhibit anti-microbial properties in some cases, which may at least partially mitigate infection risks associated with the administration of the cross-linked polymer network.

Without wishing to be bound by any particular theory, the inclusion of PDA, according to some embodiments, in the cross-linked polymer network may increase the overall mechanical strength (e.g., compressive strength) of the network by covalently interacting with both the multi-functional crosslinker and the thiolated-polymer and by forming hydrogen bonds with the amine groups associated with the thiolated-polymer. PDA may also reduce the formation time of the cross-linked polymer network (see Example 1), which can increase precision of administration as there may be limited migration and sufficient adhesion of the network prior to complete gel formation.

In some embodiments, the PDA can be synthesized from a precursor. In some embodiments, the precursor comprises dopamine hydrochloride and/or L-3,4-dihydroxyphenylalanine (L-DOPA). In some embodiments, the PDA can be synthesized to have sufficient dispersity in aqueous fluids (polydispersity index of ˜0.5) and/or reactivity with thiol, amine, and/or catechol moieties that can be present in the multi-functional crosslinker and/or the thiolated-polymer.

In some embodiments, the cross-linked polymer network comprises the adhesion promoter at a concentration. In some embodiments, the concentration of the adhesion promoter in the cross-linked polymer network is greater than or equal to 0.01% (wt/v), greater than or equal to 0.1% (wt/v), greater than or equal to 0.5% (wt/v), greater than or equal to 0.75% (wt/v), greater than or equal to 1% (wt/v), greater than or equal to 1.25% (wt/v), greater than or equal to 1.5% (wt/v), greater than or equal to 1.75% (wt/v), greater than or equal to 2% (wt/v), greater than or equal to 2.25% (wt/v), greater than or equal to 2.5% (wt/v), greater than or equal to 3% (wt/v), greater than or equal to 4% (wt/v), or greater than or equal to 5% (wt/v). In some embodiments, the concentration of PDA in the cross-linked polymer network is less than or equal to 5% (wt/v), less than or equal to 4% (wt/v), less than or equal to 3% (wt/v), less than or equal to 2.5% (wt/v), less than or equal to 2.25% (wt/v), less than or equal to 2% (wt/v), less than or equal to 1.75% (wt/v), less than or equal to 1.5% (wt/v), less than or equal to 1.25% (wt/v), less than or equal to 1% (wt/v), less than or equal to 0.75% (wt/v), less than or equal to 0.5% (wt/v), less than or equal to 0.1% (wt/v), or less than or equal to 0.01% (wt/v). Combinations of these ranges are possible (greater than or equal to 0.01% (wt/v) and less than or equal to 5% (wt/v). Other ranges are also possible.

In some embodiments, the adhesion promoter comprises a plurality of particles. The plurality of particle may promote the adhesion of the cross-linked polymer network and the bioactivity associated with the adhesion promoter. In some embodiments, the plurality of particles may have a maximum transverse dimension. In some embodiments, the plurality of particle may have a maximum transverse dimension greater than or equal to 10 nanometers, greater than or equal to 25 nanometers, greater than or equal to 50 nanometers, greater than or equal to 100 nanometers, greater than or equal to 200 nanometers, greater than or equal to 300 nanometers, greater than or equal to 400 nanometers, greater than or equal to 500 nanometers, greater than or equal to 1 micrometers, greater than or equal to 2.5 micrometers, greater than or equal to 5 micrometers. In some embodiments, the plurality of particles may have a maximum transverse dimension less than or equal to 5 micrometers, less than or equal to 2.5 micrometers, less than or equal to 1 micrometer, less than or equal to 500 nanometers, less than or equal to 400 nanometers, less than or equal to 300 nanometers, less than or equal to 200 nanometers, less than or equal to 100 nanometers, less than or equal to 50 nanometers, less than or equal to 25 nanometers, or less than or equal to 10 nanometers. Combinations of these ranges are possible (e.g., greater than or equal to 10 nm and less than or equal to 5 micrometers). Other ranges are also possible. In some embodiments, the plurality of particles comprises nanoparticles.

In some embodiments, the plurality of particles may have an average particle size. In some embodiments, the plurality of particles may have an average particle size greater than or equal to 10 nanometers, greater than or equal to 25 nanometers, greater than or equal to 50 nanometers, greater than or equal to 100 nanometers, greater than or equal to 200 nanometers, greater than or equal to 300 nanometers, greater than or equal to 400 nanometers, greater than or equal to 500 nanometers, greater than or equal to 1 micrometers, greater than or equal to 2.5 micrometers, greater than or equal to 5 micrometers. In some embodiments, the plurality of particles may have an average particle size less than or equal to 5 micrometers, less than or equal to 2.5 micrometers, less than or equal to 1 micrometers, less than or equal to 500 nanometers, less than or equal to 400 nanometers, less than or equal to 300 nanometers, less than or equal to 200 nanometers, less than or equal to 100 nanometers, less than or equal to 50 nanometers, less than or equal to 25 nanometers, or less than or equal to 10 nanometers. Combinations of these ranges are also possible (e.g., less than or equal to 5 micrometers and greater than or equal to 10 nanometers). Other ranges are also possible.

In some cases, the particles may have any of a myriad of shapes. For example, in some embodiments, the shape of the plurality of particles may comprise a sphere, a rod, a chain, a star, a flower, reef, whisker, fiber, shell, cage, and box. In other cases, the shape of the plurality of particles may comprises a sphere, disc, cylinder, rod, cube, triangle, octahedron, hexagon, pentagon, flower, platelet, cluster, etc. Different particles may independently have the same or different shapes. Other shapes and/or combinations of shapes are also possible. In some embodiments, the maximum dimension of the shapes in the aforementioned list may be nano-scale and/or micro-scale in some embodiments (e.g. a nanosphere, a nanodisc, or a nanorod), for instance, having dimensions such as those described herein.

In some embodiments, the adhesion promoter may comprise a catechol moiety, a quinone moiety, and/or a phenol moiety. Without wishing to be bound by any particular theory, the aforementioned list of moieties can enhance the adhesion of the cross-linked polymer network to the substrate (e.g., biological tissue in or on the body cavity), and in some embodiments, promote reactivity with the multi-functional crosslinker and/or the thiolated-polymer. In some embodiments, the adhesion promoter is a chemical derivative of polydopamine. In some embodiments, the adhesion promoter includes one or more of indole-5,6-quinone, eumelanin, melanin, pheomelanin, 5,6-dihydroxyindole, tyrosine, L-dopaquinone, L-leucodopachrome, serotonin, and L-dopachrome. In some embodiments, the adhesion promoter comprises indole-5,6-quinone, eumelanin, melanin, pheomelanin, 5,6-dihydroxyindole, tyrosine, L-dopaquinone, L-leucodopachrome, serotonin, and/or L-dopachrome. Other adhesion promoters may also be present, e.g., instead of or in addition to these.

In some embodiments, the adhesion promoter comprises one or more of norepinephrine, adrenaline, catecholamine, 4-(2-aminoethyl)-2-methoxyphenol hydrochloride, 5-(2-aminoethyl)pyrogallol hydrochloride, 4-methylpyrocatechol, 3-methylpyrocatechol, 3-fluoropyrocatechol, 3,4-dihydroxybenzonitrile, o-ethoxyphenol, 2-methoxy-4-methylphenol, 4-ethylpyrocatechol, 4-(aminomethyl)pyrocatechol hydrobromide, 3-methoxypyrocatechol, 3,4-dihydroxybenzyl alcohol, o-methoxybenzenethiol, 4-chloropyrocatechol, 4-tert-butylpyrocatechol, o-diethoxybenzene, 4-allyl-1,2-dimethoxybenzene, 3,4-dimethoxy-6-methylpyrocatechol, 4-phenylpyrocatechol, 2-chloro-3′,4′-dihydroxyacetophenone, sodium 2-methoxy-5-nitrophenolate, o-phenylene diacetate, o-(benzyloxy)phenol, (+-)-4-(1-hydroxy-2-(isopropylamino)ethyl)-1,2-benzenediol hydrochloride, 3,4-dihydroxybenzophenone, 3,5-di-tert-butylpyrocatechol, potassium 3,4-dihydroxybenzenesulfonate, tetrachloropyrocatechol, pyrocatechol dipentyl ether, 4-(p-nitrophenylazo) catechol, (+)-catechin, (−)-epicatechin, (+-)-p-(2-(3-(p-hydroxyphenyl)-1-methylpropylamino)ethyl)pyrocatechol hydrochloride, 4,4′-(2,3-dimethyltetramethylene)dipyrocatechol, 4-nitrocatechol sulfate dipotassium salt, disodium 4,5-dihydroxy-1,3-benzenedisulfonate (Tiron), pyrocatechol violet, caffeic acid, polycaffeic acid, tanic acid, 1,2,4-benzenetriol, pyrogallol, coniferaldehyde, 2,4-dinitrophenol, 4-nitrocatechol, and 4-methylcatechol, and/or humic acid.

As described above, the adhesive promoter may comprise catechol groups in some cases. Any suitable catechol derivative may be used to crosslink the adhesive promoter with the multi-functional crosslinker and the thiolated-polymer. In some embodiments, the catechol group comprises dopamine. Non-limiting examples of catechol derivatives contemplated herein are shown in Table 1.

TABLE 1
Exemplary catechol derivatives

| Name | Catechol derivatives |
|---|---|
| dopamine | 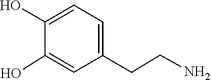 [SMILES: NCCc1ccc(O)c(O)c1] |
| 4-methyl- catechol | 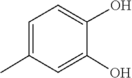 |
| DHCA | 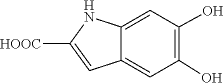 [SMILES: O=C(O)c1cc2cc(O)c(O)cc2[nH]1] |
| L-DOPA | 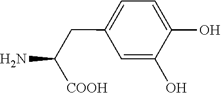 [SMILES: N[C@@H](Cc1ccc(O)c(O)c1)C(=O)O] |
| catechin | 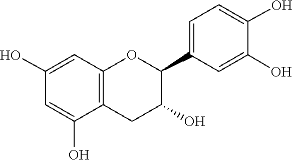 [SMILES: Oc1cc(O)c2c(c1)O[C@@H](c1ccc(O)c(O)c1)[C@H](O)C2] |
| adrenaline | 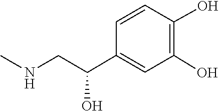 |

In some embodiments, the adhesion promoter may promote the sealing, healing, and/or occlusion of the body cavity. That is, the adhesion promoter may comprise wound healing properties that, when the cross-linked polymer network is adhered to a surface in or on the body cavity, promotes the closure of the body cavity. In some embodiments, wound healing properties may comprise antimicrobial properties, anti-inflammatory properties, and/or mechanical properties that allow for cell ingrowth and/or the formation of a cellular plug. For example, in some embodiments, the adhesion promoter, by allowing promoting adhesion of the cross-linked polymer network to the substrate, may promote cellular attachment, migration, and proliferation that can lead to closure of the body cavity.

In some embodiments, the cross-linked polymer network comprises a multi-functional crosslinker. Without wishing to be bound by any particular theory, a multi-functional crosslinker (e.g. a four-arm poly(ethylene glycol) maleimide) can promote crosslinking with the thiolated-polymer and the adhesion promoter. The multi-functional crosslinker may act as a physical crosslinker, chemical crosslinker, or a combination thereof. In certain embodiments, the multi-functional crosslinker may comprise any of myriad of polymers or copolymers that are configured to crosslink a polymer network. That is, the multifunctional crosslinker may comprise one or more functional groups, functionalized branches, and/or other chemical moieties with relatively high reactivity that, when in the presence of the thiolated-polymer and the adhesion promoter, may react to form the cross-linked polymer network. In some embodiments, the multi-functional crosslinker comprises a maleimide-functionalized crosslinker.

In some embodiments, the multi-functional crosslinker may comprise any of variety of suitable functional groups. In some embodiments, the multi-functional crosslinker comprises —NH, —NHR′. —N (R′), —SH, —OH, —COOH, —CH, —OH, —H, —PH, —PHR′, —P(R′), —CO, —NH NH, and —CHN, where R′is a hydrocarbyl group, and each R′ may be the same or different. In some embodiments, the multi-functional crosslinker may comprise an electrophilic group. In some embodiments, the electrophilic group comprises —CO Cl,—(CO)—O—(CO)—R (where R is an alkyl group), —CH═CH—CH═O and —CH═CH—C(CH)—O, halogen, —N═C═O, —N═C—S, —SOCH═CH—O(CO)—C—CH, O(CO)—C(CH)—CH—S S(CHN), —O(CO)C(CHCH)—CH, CH—CHC—NH, COON, —(CO)O N(COCH), CHO, —(CO)O N(COCH), S(O), OH, and/or N(COCH).

In some embodiments, the multi-functional crosslinker comprises multiple arms and/or branches. In some embodiments, the multi-functional crosslinker comprises at least 1, 2, 3, 4, 5, 6, 7, 8, 9, or 10 arms and/or branches and with different molecular weights ranging between greater than or equal to 1 kDa and less than or equal to 40 kDa. Without wishing to be bound by any particular theory, multi-arm polymers or copolymers, as opposed to linear polymers or copolymers, may promote crosslinking between the adhesion promoter and the thiolated-polymer as well as allow for the cross-linked polymer network to encapsulate or otherwise hold various payloads (e.g., pharmaceuticals). In some embodiments, some or all of the arms of the multi-functional crosslinker comprise at least one chemical moiety (e.g., maleimide) that is configured to react with the adhesion promoter and/or the thiolated-polymer.

In some embodiments, the multi-functional crosslinker is present in the cross-linked polymer network at a concentration. In some embodiments, the concentration of the multi-functional crosslinker in the cross-linked polymer network is greater than or equal to 0.01% wt/v, greater than or equal to 0.5% wt/v, greater than or equal to 1% wt/v, greater than or equal to 2.5% wt/v, greater than or equal to 5% wt/v, greater than or equal to 7.5% wt/v, greater than or equal to 10% wt/v, greater than or equal to 12.5% wt/v, greater than or equal to 15% wt/v, greater than or equal to 17.5% wt/v, or greater than or equal to 20% wt/v. In some embodiments, the concentration of the multi-functional crosslinker in the cross-linked polymer network is less than or equal to 20% wt/v, less than or equal to 17.5% wt/v, less than or equal to 15% wt/v, less than or equal to 12.5% wt/v, less than or equal to 10% wt/v, less than or equal to 7.5% wt/v, less than or equal to 5% wt/v, less than or equal to 2.5% wt/v, less than or equal to 1% wt/v, less than or equal to 0.5% wt/v, or less than or equal to 0.01% wt/v. Combinations of these ranges are possible (e.g., greater than or equal to 0.01% wt/v and less than or equal to 20% wt/v). Other ranges are also possible.

The multi-functional crosslinker described herein may have any one of a myriad of molecular weights. In some embodiments, the molecular weight of the multifunctional crosslinker is greater than or equal to 1 kDa, greater than or equal to 5 kDa, greater than or equal to 10 kDa, greater than or equal to 15 kDa, greater than or equal to 20 kDa, greater than or equal to 30 kDa, or greater than or equal to 40 kDa. In some embodiments, the molecular weight of the multifunctional crosslinker is less than or equal to 40 kDa, less than or equal to 30 kDa, less than or equal to 20 kDa, less than or equal to 15 kDa, less than or equal to 10 kDa, less than or equal to 5 kDa, or less than or equal to 1 kDa. Combinations of these ranges are also possible (e.g., greater than or equal to 1 kDa and less than or equal to 40 kDa). Other ranges are also possible.

The multi-functional crosslinker described herein may have any one of a myriad of compositions. In some embodiments, the maleimide-functional crosslinker comprises poly(ethylene glycol) maleimide. In some embodiments, the multi-functional crosslinker comprises maleimide, N-(2-Aminopropyl) maleimide, N-ethylmaleimide, N-phenylmaleimide, N-methylmaleimide, n-cyclohexylmaleimide, N-benzylmaleimide, N-(p-tolyl) maleimide, N-(p-tolyl) maleimide, N-(p-aminophenyl) maleimide, 1,2,3,4-cyclobutanetetracarboxdiimide, N-(p-methoxyphenyl) maleimide, N-(o-chlorophenyl) maleimide, n-cyclohexyl maleimide, N-(p-nitrophenyl) maleimide, 3,4-dibromomaleimide, N-bromomethyl-2,3-dichloromaleimide, fluoroimide, N,N′-(p-phenylene) dimaleimide, N,N′-o-phenylenedimaleimide, N,N′-m-phenylenedimaleimide, 1,6-bismalemidohexane, N-(2,4,6-trichlorophenyl) maleimide, N-(p-(phenylazo)phenyl) maleimide, N-(4-(2-benzimidazolyl)phenyl) maleimide, N-(9-acridinyl) maleimide, N-(3-fluoranthenyl) maleimide, 7-dimethylamino-3-maleimido-4-methylcoumarin, N-(4-dimethylamino-3,5-dinitrophenyl) maleimide, 2-benzylamino-N-(p-methoxyphenyl) maleimide, N, N′-(methylenedi-p-phenylene) dimaleimide, 1,1′-(methylenebis(2-ethyl-6-methyl-4,1-phenylene)) dimaleimide, N-(2-Hydroxyethyl) maleimide, N-Maleoyl-β-alanine, Alkyne-PEG4-maleimide, Dibenzocyclooctyne-maleimide, Methoxypolyethylene glycol maleimide, N-Biotinoyl-N′-(6-maleimidohexanoyl) hydrazide, Acetylene-PEG4-maleimide, DBCO-PEG4-maleimide, 5-Maleimido-fluorescein, Maleimide-PEG-OH, 2-Maleimidoethylamine trifluoroacetate salt, N-(2-Hydroxyethyl) maleimide, 1,1′-(Methylenedi-4,1-phenylene) bismaleimide, 1,1′-(Methylenedi-4,1-phenylene) bismaleimide, 1,1′-(hexane-1,6-diyl) bis(1h-pyrrole-2,5-dione), maleic anhydride, and/or poly(ethylene glycol) maleimide. In some embodiments, the multi-functional crosslinker comprises a compound described in Table 2.

TABLE 2
Select embodiments of the multi-functional crosslinker.

| | |
|---|---|
| [¹⁸F]Fluorobenzaldehyde-O-[6- (2,5-dioxo-2,5-dihydro-pyrrol-1- yl)-hexyl]oxime([¹⁸F]FBAM) | 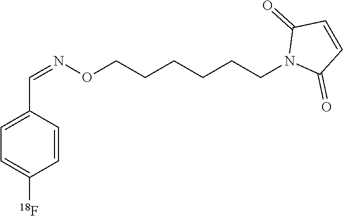 [SMILES: O=C1C=CC(=O)N1CCCCCCO/N=C\c1ccc([18F])cc1] |
| | [¹⁸F]FBAM |
| [¹⁸F]FBAM(1) | 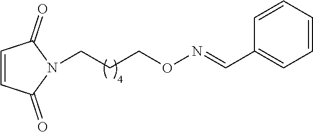 |
| | [¹⁸F]FBAM(1) |
| [¹⁸F]FBAM(2) | 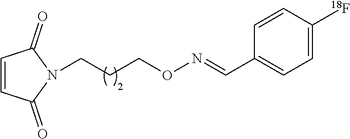 |
| | [¹⁸F]FBAM(2) |
| N-[4-[(4-[¹⁸F]fluoboenzyl- dine)aminooxy]butyl]maleimide ([¹⁸F]FBABM) | 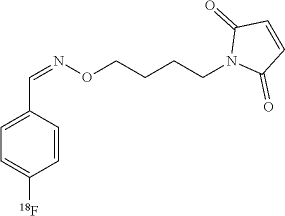 [SMILES: O=C1C=CC(=O)N1CCCCO/N=C\c1ccc([18F])cc1] |
| | [¹⁸F]FBABM |
| N-Succinimidyl-4-[¹⁸F] fluorobenzoate ([¹⁸F]SFB) | 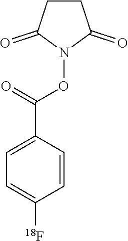 [SMILES: O=C(ON1C(=O)CCC1=O)c1ccc([18F])cc1] |
| | [¹⁸F]SFB |
| 1-[3-(2-[¹⁸F]fluoropyridin-3- yloxy)propyl]pyrrole-2,5-dione ([¹⁸F]FPyME) | 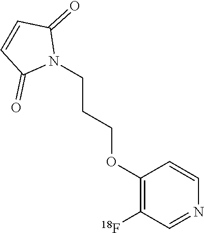 [SMILES: O=C1C=CC(=O)N1CCCOc1ccncc1[18F]] |
| | [¹⁸F]FPyME |
| [¹⁸F]Fluorobenzaldehyde-O-(2- {2-[2-(pyrrol-2,5-dione-1-yl) ethoxy]-ethoxy}-ethyl)oxime ([¹⁸F]FBOM) | 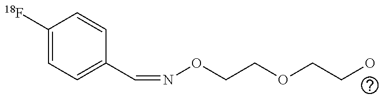 [SMILES: OCCOCCO/N=C\c1ccc([18F])cc1] |
| | [¹⁸F]FBOM |
| [¹⁸F]FBOM(4) | 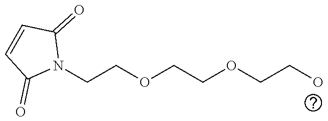 [SMILES: O=C1C=CC(=O)N1CCOCCOCCO] |
| | [¹⁸F]FBOM(4) |
| [¹⁸F]FBAM with a pyridine ([¹⁸F]FBAMPy) | 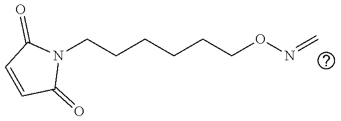 [SMILES: C=NOCCCCCCN1C(=O)C=CC1=O] |
| | [¹⁸F]FBAMPy(1) |
| [¹⁸F]FBA | 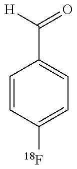 |
| | [¹⁸F]FBA |
| [¹⁸F]FPyMe(3) | 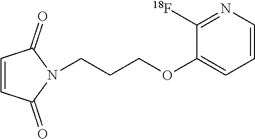 [SMILES: O=C1C=CC(=O)N1CCCOc1cccnc1[18F]] |
| | [¹⁸F]FPyMe(3) |
| [¹⁸F]FPyAM(5) | 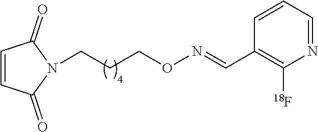 |
| | [18F]FPyAM(5) |

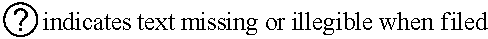

In some embodiments, the cross-linked polymer network may comprise a thiolated-polymer. Without wishing to be bound by any particular theory, the hydrophilicity and biocompatibility of the thiolated-polymer may allow the cross-linked polymer network to gel into a hydrogel, e.g., with sufficient mechanical properties to act as a plug to a body cavity and within a sufficiently short duration of time. In some embodiments, the thiolated-polymer comprises a thiol-modified gelatin (thiolated-gelatin). The thiol groups may covalently interact with the functional groups on the multi-functional crosslinker (e.g., maleimide-functionalized cross-linker) and/or the quinone moieties of the adhesion promoter (e.g., PDA), for example, to chemically cross-link with the multi-functional crosslinker and the PDA. In some embodiments, the thiolated-polymer can physically crosslink and/or chemically crosslink with the multi-functional crosslinker and the adhesion promoter. The thiolated-polymer may comprise thiolated-monomers and/or thiolated-oligomers.

The thiolated-polymer disclosed herein may comprise any of a variety of suitable compounds. In some embodiments, the thiolated-polymer comprises a thiol-modified gelatin. In some embodiments, the thiolated-polymer comprises thiolated-alginate, thiolated-polygalacturonic acid, thiolated-β-cyclodextrin, thiolated-poly(ethylene glycol), thiolated-dextran, thiolated-heparin, glutathione, dithiothreitol, and/or thiolated-glycogen. In some embodiments, the thiolated-polymer comprises a thiol-modified polypeptide (e.g., a thiol-modified protein). For example, in some embodiments, the thiolated-polymer comprises thiolated derivatives of collagen, gelatin, elastin, and/or elastin-like polypeptides such as cysteine residues. In some embodiments, the thiolated-polymer comprises a thiol-modified saccharide. As previously mentioned, the thiol groups may covalently interact with the functional groups on the multi-functional crosslinker, and accordingly, any of a myriad of thiolated-compounds may serve a similar function as the thiolated-polymer. That is, thiolated-monomers and thiolated-oligomers may serve as a suitable alternative to the thiolated-polymer (e.g., thiolated-chitosan). Other alternatives include but are not limited to thiolated-carbohydrates (e.g., thiolated-monosaccharides, thiolated-disaccharides, thiolated-oligosaccharides, and/or thiolated-polysaccharides).

In some embodiments, the thiolated-polymer is present in the cross-linked polymer network. In some embodiments, the concentration of the thiolated-polymer in the cross-linked polymer network is greater than or equal to 0.01% wt/v, greater than or equal to 1% wt/v, greater than or equal to 5% wt/v, greater than or equal to 10% wt/v, greater than or equal to 15% wt/v, greater than or equal to 20% wt/v, greater than or equal to 25% wt/v, or greater than or equal to 30% wt/v. In some embodiments, the concentration of the thiolated-polymer in the cross-linked polymer network is less than or equal to 30% wt/v, less than or equal to 25% wt/v, less than or equal to 20% wt/v, less than or equal to 15% wt/v, less than or equal to 10% wt/v, less than or equal to 5% wt/v, or less than or equal to 1% wt/v. Combinations of these ranges are possible (e.g. less than or equal to 30% wt/v and greater than or equal to 30% wt/v). Other ranges are also possible.

Other aspects of this present disclosure generally relate to a hydrogel system comprising a cross-linked polymer network. In some embodiments, the hydrogel system comprises the cross-linked polymer network. The cross-linked polymer network, in some embodiments, comprises properties that may be able to reshape, close, occlude, and/or promote healing of the body cavity (e.g., a fistula). That is, in some cases, the cross-linked polymer network may serve as a scaffold for cell in growth such that the network can, over time, be degraded and replaced by a cell-plug.

In some embodiments, the cross-linked polymer in configured to release the adhesion promoter over a period of time. In some embodiments, the cross-linked polymer is configured to release the PDA over a period of time. In some embodiments, the cross-linked polymer is configured to release the adhesion promoter for at least 24 hours, at least 48 hours, at least 72 hours, at least 100 hours, at least 200 hours, at least 300 hours, at least 400 hours, or at least 500 hours. Without wishing to be bound by any particular theory, sustained release of PDA into a body cavity may promote healing of the body cavity and potentially act as an anti-bacterial component.

In some embodiments, the cross-linked polymer network can degrade over a period of time. To further promote healing and/or closure of the body cavity (e.g., a fistula), the cross-linked polymer network may advantageously degrade over time. The rate at which the cross-linked polymer network degrades can, in some embodiments, correspond to the mass remaining after a period of time exposed to collagenase present in an amount of 2.5 U/ml. In some embodiments, the mass of the cross-linked polymer network after 30 days of exposure to collagenase, present in an amount of 2.5 U/ml, is greater than or equal to 50%, greater than or equal to 60%, greater than or equal to 70%, greater than or equal to 80%, or greater than or equal to 90% of the original mass of the cross-linked polymer network prior to exposure to the collagenase. In some embodiments, the mass of the cross-linked polymer network after 30 days of exposure to collagenase, present in an amount of 2.5 U/ml, is less than or equal to 90%, less than or equal to 80%, less than or equal to 70%, less than or equal to 60%, or less than or equal to 50% of the original mass of the cross-linked polymer network prior to exposure to the collagenase. Combinations of these ranges are possible (e.g., greater than or equal to 50% and less than or equal to 90%). Other ranges are also possible.

In some embodiments, the cross-linked polymer network has relatively high compressive strength. In some embodiments, the compressive strength of the cross-linked polymer network is greater than or equal to 25 kPa, greater than or equal to 35 kPa, greater than or equal to 45 kPa, greater than or equal to 55 kPa, greater than or equal to 65 kPa, greater than or equal to 75 kPa, greater than or equal to 85 kPa, etc. In some embodiments, the compressive strength of the cross-linked polymer network is less than or equal to 85 kPa, less than or equal to 75 kPa, less than or equal to 65 kPa, less than or equal to 55 kPa, less than or equal to 45 kPa, less than or equal to 35 kPa, or less than or equal to 25 kPa. Combinations of these ranges are also possible (e.g., less than or equal to 25 kPa and less than or equal to 85 kPa). Other ranges are also possible.

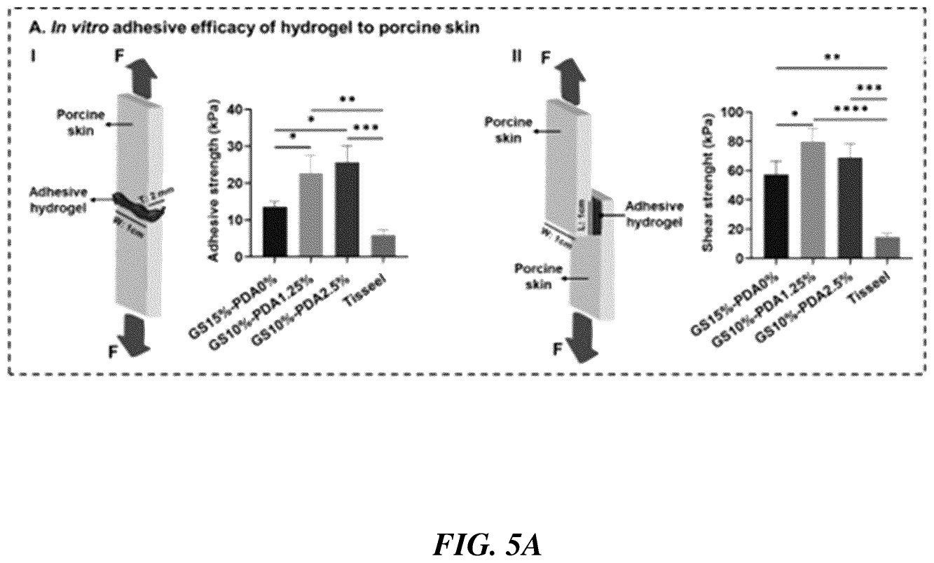

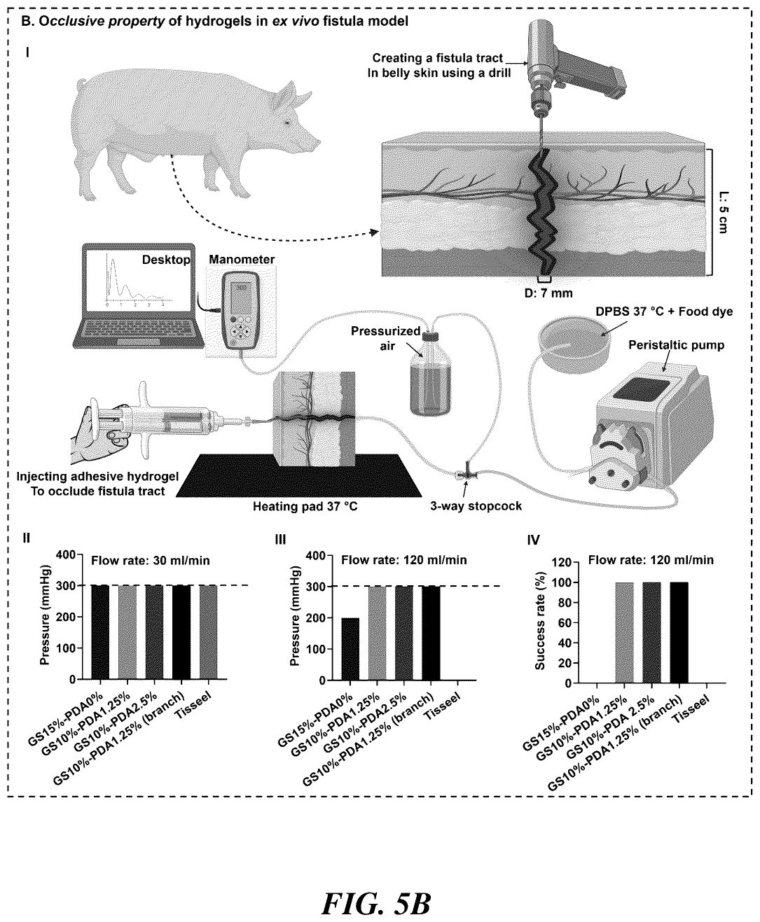

In some embodiments, the cross-linked polymer network has a relatively high adhesive strength. In some embodiments, the adhesive strength of the cross-linked polymer network is greater than or equal to 5 kPa, greater than or equal to 10 kPa, greater than or equal to 15 kPa, greater than or equal to 20 kPa, greater than or equal to 25 kPa, or greater than or equal to 30 kPa when measured by using the method described in Example 1 and FIG. 5A(I). In some embodiments, the adhesive strength of the cross-linked polymer network is less than or equal to 30 kPa, less than or equal to 25 kPa, less than or equal to 20 kPa, less than or equal to 15 kPa, less than or equal to 10 kPa, or less than or equal to 5 kPa when measured by using the method described in Example 1 and FIG. 5A(I). Combinations of these ranges are possible (e.g., less than or equal to 5 kPa and less than or equal to 30 kPa). Other ranges are also possible.

In some embodiments, the cross-linked polymer network has a relatively high adhesive shear strength. In some embodiments, the cross-linked polymer network has an adhesive shear strength of greater than or equal to 40 kPa, greater than or equal to 50 kPa, greater than or equal to 60 kPa, greater than or equal to 70 kPa, greater than or equal to 80 kPa, or greater than or equal to 90 kPa when measured by using the method described in Example 1 and FIG. 5A(II). In some embodiments, the cross-linked polymer network has an adhesive shear strength of less than or equal to 90 kPa, less than or equal to 80 kPa, less than or equal to 70 kPa, less than or equal to 60 kPa, less than or equal to 50 kPa, or less than or equal to 40 kPa. Combinations of these ranges are possible (e.g. less than or equal to 90 kPa and less than or equal to 40 kPa) when measured by using the method described in Example 1 and FIG. 5A(II). Other ranges are also possible.

In some embodiments, the cross-linked polymer network has a storage modulus that increases at the onset of gelation. In some embodiments, the cross-linked polymer network has a storage modulus that is greater than or equal to 1 kPa, greater than or equal to 2.5 kPa, greater than or equal to 5 kPa, greater than or equal to 7.5 kPa, greater than or equal to 10 kPa, greater than or equal to 12.5 kPa at the onset of gelation. In some embodiments, the cross-linked polymer network has a storage modulus that is less than or equal to 12.5 kPa, less than or equal to 10 kPa, less than or equal to 7.5 kPa, less than or equal to 5 kPa, less than or equal to 2.5 kPa, or less than or equal to 1 kPa at the onset of gelation. Combinations of these ranges are possible (e.g., less than or equal to 10 kPa and less than or equal to 1 kPa). Other ranges are also possible.

In some embodiments, the cross-linked polymer network forms a gel within a relatively short duration of time. In some embodiments, the cross-linked polymer network has a gel formation time greater than or equal to 1 second, greater than or equal to 2 seconds, greater than or equal to 3 seconds, greater than or equal to 4 seconds, greater than or equal to 5 seconds, greater than or equal to 10 seconds, or greater than or equal to 20 seconds. In some embodiments, the cross-linked polymer network has a gel formation time less than or equal to 1 seconds, less than or equal to 2 seconds, less than or equal to 3 seconds, less than or equal to 4 seconds, less than or equal to 5 seconds, less than or equal to 10 seconds, or less than or equal to 20 seconds. Combinations of these ranges are possible (e.g., less than or equal to 1 second and greater than or equal to 10 second). Other ranges are also possible.

In some embodiments, the cross-linked polymer network can withstand relatively high pressure. To effectively seal a body cavity (e.g., a fistula), the cross-linked polymer may be able to withstand relatively high pressures without being dislodged or displaced. In some embodiments, the cross-linked polymer network can withstand pressure of less than or equal to 300 mmHg at flow rate of 30 ml/min, and 120 ml/min in ex vivo fistula model, as depicted in Example 1, FIG. 5B(II), and FIG. 5B(III). In some embodiments, the cross-linked polymer network can withstand a pressure greater than or equal to 10 mmHg, greater than or equal to 25 mmHg, greater than or equal to 50 mmHg, greater than or equal to 100 mmHg, greater than or equal to 150 mmHg, greater than or equal to 200 mmHg, greater than or equal to 250 mmHg, greater than or equal to 300 mmHg, greater than or equal to 350 mmHg, greater than or equal to 400 mmHg, greater than or equal to 450 mmHg, or greater than or equal to 500 mmHg at flow rate of 120 ml/min in ex vivo fistula model, as depicted in Example 1, FIG. 5B(II), and FIG. 5B(III). In some embodiments, the cross-linked polymer network can withstand a pressure less than or equal to 500 mmHg, less than or equal to 450 mmHg, less than or equal to 400 mmHg, less than or equal to 350 mmHg, less than or equal to 300 mmHg, less than or equal to 250 mmHg, less than or equal to 200 mmHg, less than or equal to 150 mmHg, less than or equal to 100 mmHg, less than or equal to 50 mmHg, less than or equal to 25 mmHg, or less than or equal to 10 mmHg at flow rate of 120 ml/min in ex vivo fistula model, as depicted in Example 1, FIG. 5B(II), and FIG. 5B(III). Combinations of these ranges are possible (e.g., greater than or equal to 10 mmHg and less than or equal to 500 mmHg).

In some embodiments, the cross-linked polymer further comprises anti-inflammatory compounds, anti-inflammatory peptides, and/or growth factors that accelerate the healing of the body cavity. In some embodiments, the anti-inflammatory compounds and/or peptides that accelerate the healing of the body cavity comprise pentadecapeptide BPC 157, cyclo-gly-pro (cyclic peptide), and/or prostaglandin. In some embodiments, the growth factors comprise epidermal growth factor, transforming growth factor, vascular endothelial growth factor, fibroblast growth factor, platelet derived growth factor, and/or keratinocyte growth factor. Other growth factors are also possible. In some embodiments, the cross-linked polymer further comprises pentadecapeptide BPC 157. The inclusion of these components may, without wishing to be bound by any particular theory, allow for the body cavity (e.g., a fistula) to heal and/or close in a more efficient manner, and potentially with reduced risk of infection by reducing local inflammation and local bacterial load, in some embodiments. Accordingly, the cross-linked polymer network, in some embodiments, not only promotes healing and/or closure of the body cavity (e.g., fistula), but may also allow for the controlled and sustained release of compounds (e.g., anti-inflammatory compounds) that promote body cavity healing and/or closure.

In some embodiments, the cross-linked polymer network further comprises a contrast agent. For example, it may be desirable and/or advantageous to monitor the progress and/or healing status of a fistula in a subject, or whether the cross-linked polymer network has dislodged and/or migrated from its original location. Accordingly, the contrast agent (e.g., a radiopaque agent) may allow for the cross-linked polymer network to be visible using any of a myriad of medical imaging technologies (e.g., sonography, radiography, computed tomography (CT)) and/or magnetic resonance imaging (MRI). In some embodiments, the contrast agent comprises iohexol. In some embodiments, the contrast agent comprises iohexol, iopromide, ioversol, iodixanol, ioxilan, iopamidol, lipiodol, Dotarem (gadoterate meglumine), Evoist (gadoxetate disodium), Gadavist (gadobutrol), Magnevist (gadopentetate dimeglumine), and/or Multihance (gadobenate dimeglumine). In some embodiments, the contrast agent is non-ionic or ionic compound. In some embodiments, the contrast agent is hydrophilic or hydrophobic. In some embodiments, the contrast agent comprises a catechol and/or quinone moiety (e.g., polydopamine) for ultra-sound imaging. In some embodiments, the contrast agent comprises polydopamine. In some embodiments, the contrast agent comprises any of a myriad of compounds that promote contrast when viewed using a medical imaging system. In some embodiments, the cross-linked polymer network comprises the contrast agent in an amount greater than or equal to 20% v/v, greater than or equal to 30% v/v, greater than or equal to 40% v/v, or greater than or equal to 50% v/v. In some embodiments, the cross-linked polymer network comprises the contrast agent in an amount less than or equal to 50% v/v, less than or equal to 40% v/v, less than or equal to 30% v/v, or less than or equal to 20% v/v. Combinations of these ranges are possible (e.g., less than or equal to 20% v/v and greater than or equal to 50% v/v). Other ranges are also possible.

In some embodiments, the cross-linked polymer network further comprises a pharmaceutical agent. In some embodiments, the pharmaceutical agent comprises an antifungal agent. In other embodiments, the pharmaceutical agent comprises an antimicrobial compound, for example, to prevent infection within a fistula. In some embodiments, the antimicrobial compound comprises a penicillin. Non-limiting examples include penicillin V, penicillin G, amoxicillin, amoxicillin/clavulonate, ampicillin, nafcillin, oxacillin, dicloxacillin, piperacillin, pipercillin/tazobactam, and the like. In some embodiments, the antimicrobial compound comprises a macrolide. Examples include, but are not limited to, azithromycin, clarithromycin, fidaxomicin, erythromycin, telithromycin, and the like. In some embodiments, the antimicrobial compound comprises a cephalosporin. Examples include, but are not limited to, cefacetril, cefradin, cefroxadin, cefaloglycin, cefaclor, cefalexin, cefadroxil, cefatrizin, cefazedon, cefapirin, ceftezol, cefazolin, cefazaflur, cefalotin, cefaloridine, cefalonium, and the like. In some embodiments, the antimicrobial compound comprises a fluoroquinolone. Examples include balofloxacin, grepafloxacin, levofloxacin, pazufloxacin, sparfloxacin, temafloxacin, clinafloxacin, gatifloxacin, moxifloxacin, sitafloxacin, prulifloxacin, besifloxacin, delafloxacin, and the like. In some embodiments, the antimicrobial compound comprises a beta-lactam.

Examples include penams, carbapenams, clavams, penems, carbapenems, cephems, carbacephems, oxacephems, monobactams, and the like. Combinations are also possible (e.g., the coating may comprise a penicillin and a beta-lactam or a fluoroquinolone and a cephalosporin, etc.).

Another aspect of the present disclosure generally relates to methods of forming a cross-linked polymer network. While the formation of the cross-linked polymer network may advantageously occur within the body cavity, the present disclosure is not limiting in that manner. Accordingly, the formation of the cross-linked polymer network may occur, in some embodiments, in any of a myriad of environments including, but not limited to, in a reaction vessel (e.g., a beaker or petri dish) and/or on an inorganic surface (e.g., a glass slide). The formation of the cross-linked polymer network is characterized, in some embodiments, by the time at which the storage modulus is approximately equivalent to the loss modulus as apparent during rheological measurements (Example 1).

In some embodiments, the cross-linked polymer network is formed by combining the first fluid and the second fluid. In some embodiments, combining the first and the second fluid produces a mixture comprising the first and the second fluid. In certain embodiments, combining the first and second fluid comprises physically contacting the first fluid with the second fluid and/or vice versa. Accordingly, in some embodiments, the cross-linked polymer network forms upon physical contact between the first and the second fluid. That is, physical contact between the first fluid and the second fluid, in some embodiments, initiates a reaction that forms the cross-linked polymer network. In certain instances, combining the first and second fluid comprises mixing, stirring, agitating, dispensing, and/or interspersing the first fluid and the second fluid. For example, the first fluid and the second fluid may be combined via dispensing from a device such as a dual-barrel syringe, where the first fluid is placed in a first reservoir and the second fluid is placed in a second reservoir, such that the first and second fluid physically contact each other after exiting from the syringe. In some embodiments, the dual barrel syringe comprises a needle having any one of a myriad of gauges, a mixing tip, one or more dual-lumen catheters, and/or a nozzle to create a spray of the first and second fluids. In some embodiments, the dual-barrel syringe allows for the administration of the first and second fluid to a subject.

In some embodiments, the first fluid comprises a first solution. That is, the first fluid, in certain instances, may comprise solutes (e.g., PDA, PEM, and/or the thiolated-polymer) that are at least partially dissolved in a solvent. In some embodiments, the first fluid comprises a dispersion, suspension, colloid, and/or emulsion. That is, the first fluid may comprise undissolved and/or suspended components and/or one or more compositional domains. In some embodiments, the second fluid comprises a second solution. That is, the second fluid, in certain instances, may comprise solutes (e.g., PDA, PEM, and/or the thiolated-polymer) that are at least partially dissolved in a solvent. In some embodiments, the second fluid comprises a dispersion, suspension, colloid, and/or emulsion. That is, the second fluid may comprise undissolved and/or suspended components and/or one or more compositional domains. In some embodiments, the first fluid comprises the adhesion promoter and the multi-functional crosslinker. In some embodiments, the second fluid comprises the thiolated-polymer. The first fluid, the second fluid, or both comprise, in some cases, the anti-inflammatory compounds, peptides that accelerate the healing of the body cavity, the contrast agent, and/or the pharmaceutical agent.

According to some embodiments, a mixture comprising the first fluid and the second fluid is administered to form a cross-linked network in a body cavity. To effectively treat a wound, such as a body cavity or fistula, the mixture may be dispensed into the body cavity in manner that allows the first and the second fluid to physically contact each other thereby forming the cross-linked polymer network. In some embodiments, administering the mixture comprises dispensing, injecting, providing, spraying and/or delivering the mixture into the body cavity (e.g., a fistula) or on the surface of a wound (e.g., a fistula). Upon administering the mixture into the body cavity or a short period of time thereafter, the mixture may crosslink to form the cross-linked polymer network within the body cavity. In some embodiments, the mixture is administered to treat a condition or ailment (e.g., an anal fistula).

In some embodiments, the administered mixture, which forms the cross-link polymer network, adheres to the body cavity and/or to an interior surface of the body cavity. The presence of the adhesion promoter within the cross-linked polymer network may mediate the adhesion between the cross-linked polymer network and the body cavity thereby sealing the body cavity upon administration. In some embodiments, the cross-linked polymer forms a plug within the body cavity thereby sealing the body cavity. In certain cases, the cross-linked polymer, formed by the administered mixture, adheres to wet and/or irregular surface within the body cavity with sufficient adhesion to prevent dislodgement and/or migration of the plug.

In some embodiments, the body cavity comprises an abnormal opening, void, or other lack of biological tissue in an area of an organism that should have biological tissue. In some embodiments, the body cavity comprises a fistula (e.g., an anal fistula). However, the present disclosure is not intended to be limiting in this manner, and the body cavity may comprise any of myriad of body cavities including but not limited to blood vessels, aneurysms, tooth cavities, diabetic ulcers, vascular malformation, lymph vessels, and vascular shunts, among others.

The mixture, according to some embodiments, is administered into a body cavity in a subject. In some embodiments, the subject is in need (e.g., need of treatment for a fistula). In some embodiments, the subject comprises a living organism. In some embodiments, the subject comprises a non-living organism. According to certain embodiments, the subject is a human. The subject may also be a non-human mammal in some cases. The subject may need and/or desire the administration of the mixture to treat physiological ailments associated with the body cavity.

The mixture may be administered to the subject using any of a myriad of devices. In some embodiments, the mixture is administered using a double-barrel syringe as previously described in this disclosure. The mixture may also be administered using a syringe, or more than one syringe in some cases. In some embodiments, the mixture is administered using a catheter, such as a double-lumen catheter, a mixing tip, and/or a needle, or via spraying of the mixture. The first fluid and the second fluid may also be administered in the body cavity using separate single-lumen catheters to form the cross-linked polymer network within the body cavity.

In some embodiments, after the mixture is administered in the body cavity, closure of the body cavity may be promoted. In some embodiments, the body cavity may have a scratch shrinkage area greater than or equal to 15%, greater than or equal to 20%, greater than or equal to 25%, greater than or equal to 30%, greater than or equal to 35%, greater than or equal to 40%, greater than or equal to 45%, or greater than or equal to 50% after 12 hours. In some embodiments, the body cavity may have a scratch shrinkage area less than or equal to 50%, less than or equal to 45%, less than or equal to 40%, less than or equal to 35%, less than or equal to 30%, less than or equal to 25%, less than or equal to 20%, or less than or equal to 15% after 12 hours. Combinations of these ranges are possible (e.g., greater than or equal to 15% and less than or equal to 50%). Other ranges are possible. Scratch shrinkage area may be determined by making a scratch in a well with a sterilized cell scrapper, and growing cells in the well to facilitate the closure of the scratch. The change in area of the scratch may be measure via microscopy techniques. More information of the wound-closure properties of the cross-linked polymer network can be found in Example I and Example II.

In some embodiments, the cross-linked polymer network exhibits a percentage of bacterial growth inhibition. In some embodiments, the cross-linked polymer network has an anti-bacterial greater than or equal to 25% bacterial growth inhibition, greater than or equal to 30% bacterial growth inhibition, greater than or equal to 35% bacterial growth inhibition, greater than or equal to 40% bacterial growth inhibition, greater than or equal to 45% bacterial growth inhibition, greater than or equal to 50% bacterial growth inhibition, greater than or equal to 55% bacterial growth inhibition, greater than or equal to 60% bacterial growth inhibition, greater than or equal to 65% bacterial growth inhibition, greater than or equal to 70% bacterial growth inhibition, greater than or equal to 75% bacterial growth inhibition, greater than or equal to 80% bacterial growth inhibition, greater than or equal to 85% bacterial growth inhibition, or greater than or equal to 90% bacterial growth inhibition, after 12 hours of incubation. In some embodiments, the cross-linked polymer network has an anti-bacterial less than or equal to 90% bacterial growth inhibition, less than or equal to 85% bacterial growth inhibition, less than or equal to 80% bacterial growth inhibition, less than or equal to 75% bacterial growth inhibition, less than or equal to 70% bacterial growth inhibition, less than or equal to 65% bacterial growth inhibition, less than or equal to 60% bacterial growth inhibition, less than or equal to 55% bacterial growth inhibition, less than or equal to 50% bacterial growth inhibition, less than or equal to 45% bacterial growth inhibition, less than or equal to 40% bacterial growth inhibition, less than or equal to 35% bacterial growth inhibition, less than or equal to 30% bacterial growth inhibition, or less than or equal to 25% bacterial growth inhibition, after 12 hours of incubation. Combinations of these ranges are possible (e.g., greater than or equal to 25% bacterial growth inhibition and less than or equal to 90% bacterial growth inhibition). Other ranges are possible.

The antibacterial property effect of the cross-linked polymer network may be determined against E.coli based on the zone of inhibition method. The optical density (OD) of the bacterial inoculum was measured at 600 nm using a Nanodrop device (Fisher Scientific PA, USA) and adjusted to 0.2 by dilution in Bacto tryptic soy broth (Fisher Scientific PA, USA). Afterward, 150 μl of the diluted bacterial inoculum was uniformly spread onto sterile tryptic soy agar plates (Fisher Scientific PA, USA) using a cell spreader. The cross-linked polymer network was then positioned on the agar plates. For comparison, a positive control may be established by including a filter paper containing 20 ul of Ciprofloxacin solution (8 mg/ml) on each agar plate.

Subsequently, the plates may be incubated at 37° C. for 12 hours. The clear zone surrounding each sample can be measured, and the area of inhibition can be calculated. This measurement may then be compared with the positive control (Ciprofloxacin) to determine the percentage of bacterial growth inhibition.

The following examples are intended to illustrate certain embodiments of the present invention, but do not exemplify the full scope of the invention.

### EXAMPLE 1

### Introduction

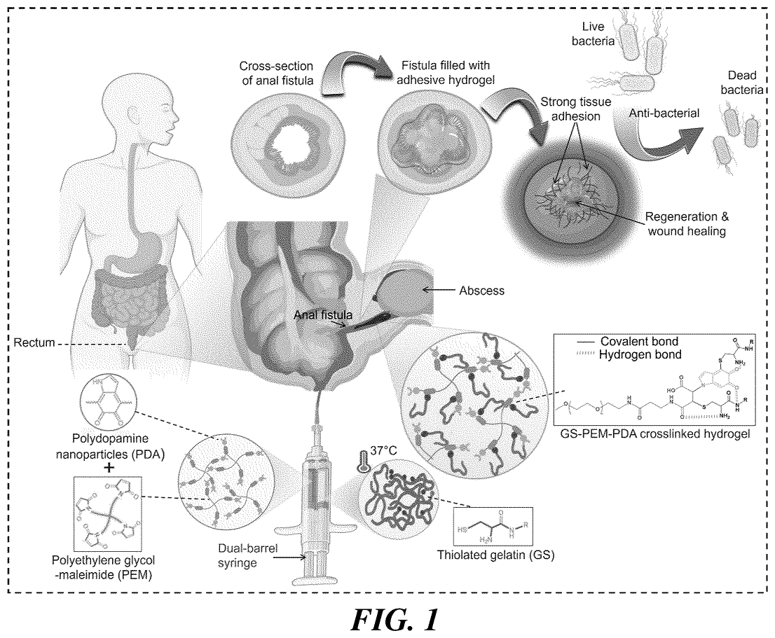

To improve the treatment of life-threatening fistulas, a minimally invasive bioadhesive platform was developed in this example. This platform was designed to seal and/or close fistula tracts using an injectable, antibacterial, and regenerative hydrogel with relatively high mechanical strength. A modification of dopamine hydrochloride to its more reactive nanosize form, referred to as polydopamine nanoparticles (PDA), was shown to improve the hydrogel's bioactivity and adhesion properties. By combining modified gelatin (thiolated-gelatin, GS) and 4-arm poly(ethylene glycol) maleimide (PEM, MW=20 kDa) as a crosslinker, a biocompatible, durable, antimicrobial, wound healing promoting adhesive was formed, which could be used to occlude fistula tracts (FIG. 1).

The adhesive, wound healing, and antimicrobial properties of PDA, when combined with cell friendly motifs of GS, can be a suitable material for occlusion and reshaping of fistulas, as this example demonstrates. A dual crosslinking mechanism of thiol groups with maleimide and quinone moieties of PDA can improve mechanical and adhesion properties of the hydrogel, reducing the likelihood of dislodgement of the hydrogel from fistulous tracts. This bioengineering approach may reduce morbidity and mortality by successfully occluding and healing anal fistulas, improving the quality of life of the patient.

As the mechanical efficiency of hydrogels is generally challenging, extensive in vitro mechanical testing such as rheometry, compression test, adhesion and fistula bench test were performed to evaluate mechanical resistance of the hydrogel in this example under pressure and shear stress applied in physiological conditions. Results of these tests were compared with the commercial fibrin glues such as Tisseel by Baxter. Moreover, to understand and improve the efficacy and durability of the hydrogel inside the fistula tract, physical characterization such as swelling behavior in DPBS (37° C.), degradation profile in the presence of collagenase (2.5 U/ml) was studied in this example, and the release profile of PDA from the hydrogels was estimated.

Fistula tracts also present complex micro-environments, which pose challenges for spontaneous healing and increase the possibility of recurrences. Factors like bacterial colonization, chronic inflammation, and tract epithelization may further hinder spontaneous recovery. Consequently, the cytocompatibility, antibacterial activities, and regenerative properties of the hydrogels may play an important role in facilitating fistula tract closure under some conditions. Given the persistent challenge of bacterial recurrence within the fistula tract, an evaluation was conducted of the proposed hydrogel's antibacterial effectiveness. This assessment was used in this example to address a significant aspect of fistula treatment. Moreover, a scratch assay was performed to evaluate proliferation and migration of the skin fibroblast cells in the presence of elutes collected from GS-PEM-PDA containing varying concentrations of PDA (0%, 1.25% and 2.5% wt/v).

### Results and Discussion

### Synthesis and Characterization of Polydopamine Nanoparticles (PDA)

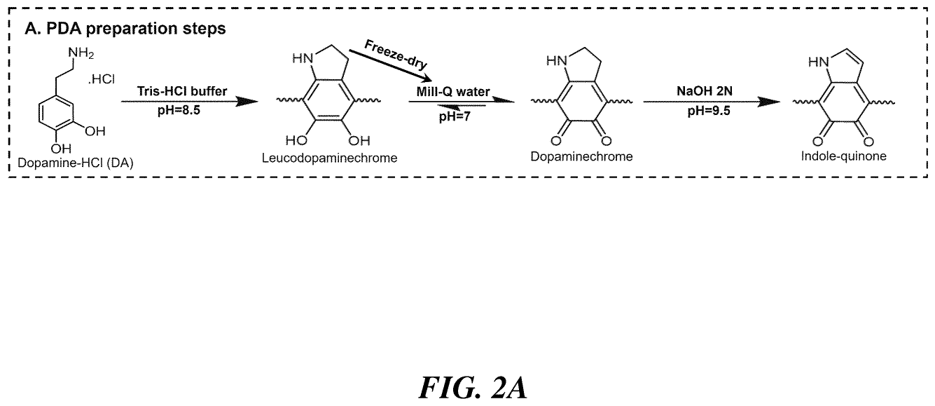

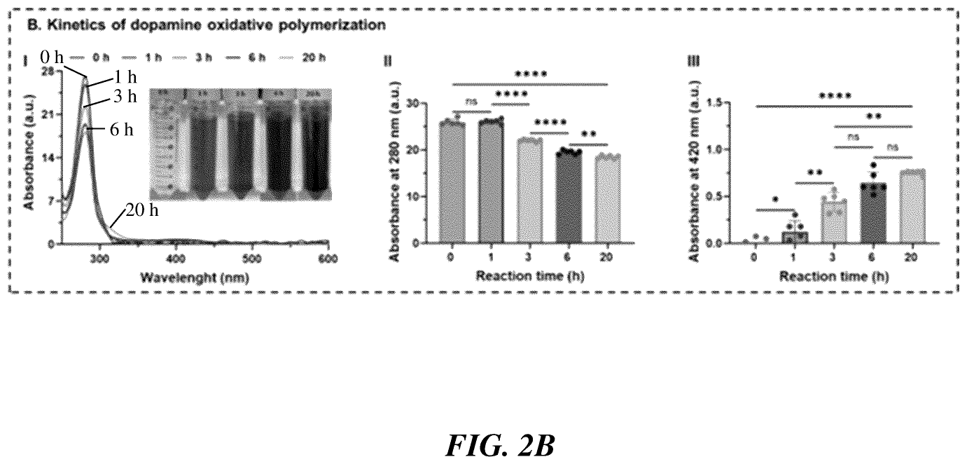

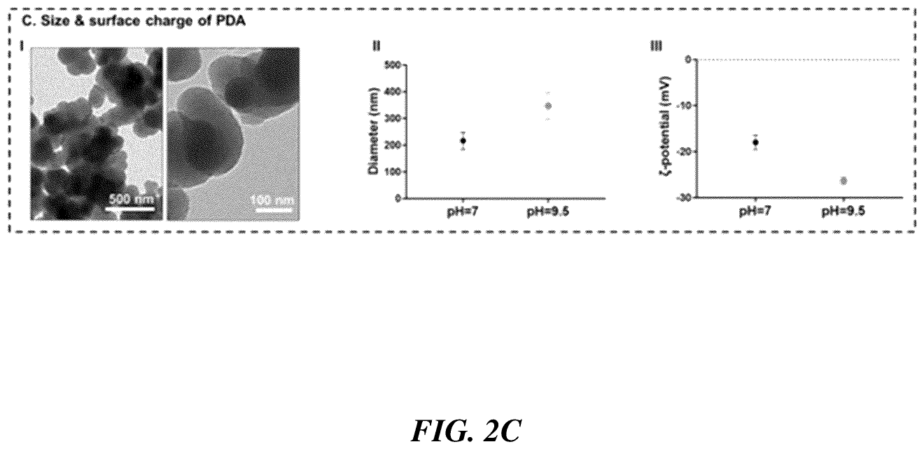

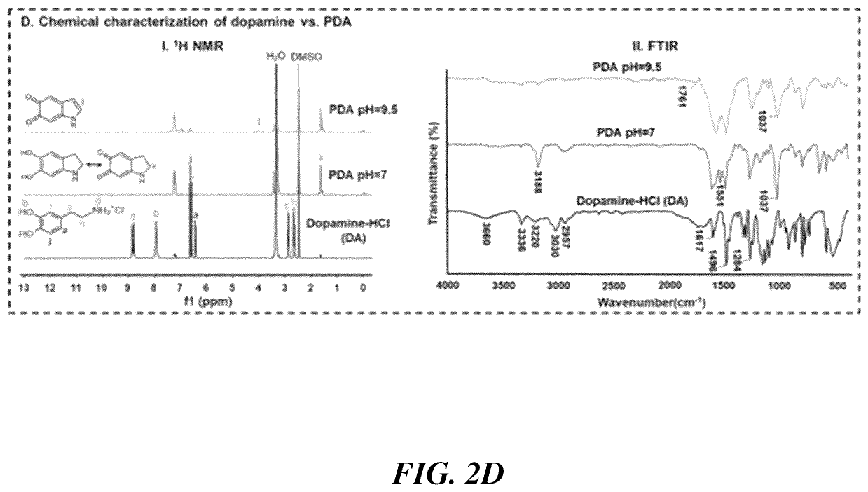

Dopamine can be converted into PDA through polymerization process involving pi-pi (π-π) stacking and hydrogen bonding. These conversions take place under alkaline conditions in the presence of oxygen. PDA can be a versatile adhesive to the surface of tissue in wet environment through hydroxyl groups of catechol or carbonyl groups of quinone. In this section of the example, the objective was to synthesize PDA with enhanced solubility and ensure the majority of the PDA exist in their quinone form with more reactivity to participate in covalent interactions with thiol, amine or catechol moieties. This can lead to relatively strong adhesion properties and mechanical strength of the hydrogel. PDA was synthesized from dopamine hydrochloride (dopamine-HCl). FIG. 2A schematically illustrates polymerization of dopamine-HCl (DA) and its potential structural changes following pH adjustments. To produce PDA, DA self-polymerized in an alkaline environment using Tris-HCl buffer (pH=8.5). The resultant PDA were washed with Milli-Q water, freeze dried and stored at room temperature until used to prevent aggregation. Prior to use, the freeze-dried PDA were dispersed in Milli-Q water at pH=7 and the pH was gradually raised to 9.5 using sodium hydroxide (NaOH, 2N) to achieve sufficient dispersity and minimize agglomeration.

To monitor the kinetics of DA oxidative polymerization in Tris-HCl buffer, UV-Vis spectra of the solution across 250-600 nm was obtained as the synthesis time progressed (FIG. 2B(I)). When 2 mg/ml of DA was dissolved in Tris-buffer, a strong UV absorption band was detected at 280 nm. This absorption can be attributed to the transitions of the amino, phenolic, and aromatic components in the DA molecule, specifically the pi-pi (π-π) and n-pi (π) transitions. A new broad peak emerged with a maximum absorbance at 420 nm after a reaction time of 1 h, indicating gradual development of PDA. Notably, there was a gradual change in the color of the DA solution during polymerization from colorless to a dark brown color (opaque in FIG. 2B(I)) over time (top right inset in FIG. 2B(I)), and precipitates were observed after centrifugation. As can be seen in FIG. 2B(II & III) while the absorbance peak at 280 nm decreased, the peak at 420 nm rose over time, representing conversion of DA to PDA.

FIG. 2C(I) depicts transmission electron microscopy (TEM) images of PDA produced in Tris-HCl buffer. The left image shows a cluster of PDA, and the right image provides a closer look at the structural details. The PDA exhibited irregular shapes with smooth edges and had an average size of ˜200 nm. Some agglomeration was observed due to the powder-like consistency of the nanoparticles in their dry state. Additionally, dynamic light scattering (DLS) (FIG. 2C(II)) showed the average hydrodynamic size of ˜220 nm in the liquid state at PH=7 and slightly larger size of ˜340 nm at pH=9.5 with the polydispersity index (PDI) of ˜0.5. This indicated that the sample was uniformly dispersed in the aqueous solution. PDA is typically larger in size when assessed by DLS as compared with TEM. This difference arises because PDA are surrounded by water molecules during DLS measurements, and their size is estimated in the swollen state, while for TEM analysis, PDA are dried. FIG. 2C(III) compares the surface charge of PDA at neutral and alkaline pH. An increase in the pH from 7 to 9.5 resulted in a decrease in zeta (zeta)-potential from −18 to −26 mV. Without wishing to bound to any particular theory, surface charges generally above +20 mV or generally below −20 mV can be associated with enhanced stability, and suggests that PDA exhibits greater structural stability at pH=9.5 compared to pH=7.

The chemical structure of DA versus PDA at different pH was studied by ¹H NMR (FIG. 2D(I)). In DA form, two significant peaks between 2.5 and 3 ppm (see peaks h and c), represent the methylene groups of DA. The aromatic protons appeared in the range of 6.3 to 6.7 ppm (see peaks j, i and a), and two peaks at 8 ppm and 9 ppm are related to O—H and N—H and groups, respectively (see peaks b and d). After the oxidative self-polymerization took place, the methylene peaks weakened, and the ring methylene proton CH₂—N peak at 1.6 ppm were strengthened in the spectra of PDA (see peak k). In addition, the peaks associated with aromatic protons changed most likely due to oxidation and crosslinking. Phenolic and amine peaks (labeled as b and d) in DA spectrum were weakened in PDA spectra. By increasing pH from 7 to 9.5, a small peak at 4 ppm started to appear in the PDA spectrum that is related to the C═C—H bond in the pentagonal ring (see peak 1), suggesting formation of indole structure at pH=9.5.

The structural changes of DA to PDA were also confirmed by FTIR (FIG. 2D(II)). The peaks observed in the FTIR spectrum of DA were at 3660 cm⁻¹ (free O—H groups), 3336 cm⁻¹ (amine N—H stretching), 3220 cm⁻¹ (phenol O—H stretching), 3030 cm⁻¹ (aromatic C—H stretching), 2957 cm⁻¹ (alkyl C—H stretching), 1617 cm⁻¹ (amine N—H bending), 1496 cm⁻¹ (aromatic C═C stretching), and 1284 cm⁻¹ (amine C—N stretching). After oxidative polymerization of DA to PDA, several peaks in the area of 4000-2700 diminished. Specifically, formation of dihydroxyindoline moiety of PDA (i.e., leucodopaminechrome) at pH=7, most likely resulted in the stretching vibrations of O—H and N—H at 3188 cm⁻¹ and 1551 cm⁻¹ respectively. The strong shoulder band at 1761 cm⁻¹ appeared after increasing the pH of PDA to 9.5 can be correlated to the C—O bonds in quinone structure. Furthermore, the peak at 1037 cm⁻¹ can be attributed to C—N bonds in the pentagonal ring of PDA. The changes in the signature peaks of the FTIR spectra of PDA suggest successful oxidative polymerization of DA.

### Hydrogel Formation Kinetic and its Tunable Rheological Properties

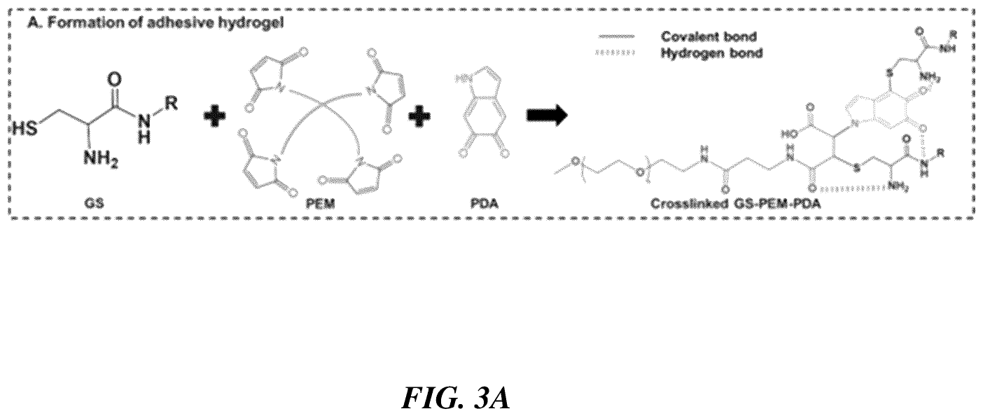

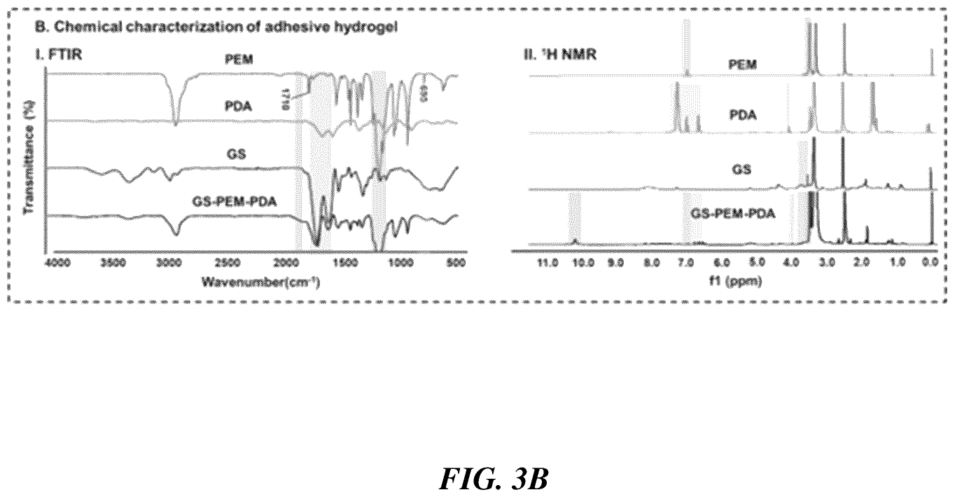

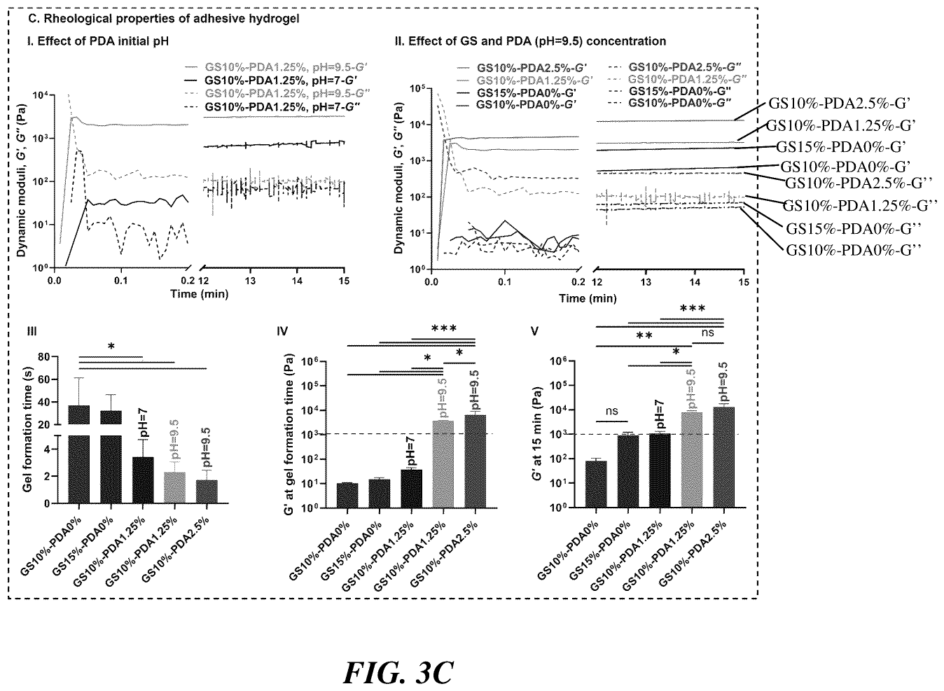

In this example, to prepare the hydrogels, a four-arm PEG maleimide (PEM), as opposed to a linear PEG, was utilized to improve crosslinking functionality and improve the efficiency of encapsulating various cargos (e.g., PDA) or drugs for delivery purposes. As shown in FIG. 3A, the crosslinking between GS and PEM takes place through a Michael addition reaction. Without wishing to be bound by any particular theory, in this process, the thiol groups in GS react with the C═C bond within the maleimide ring, leading to the formation of thioosuccinimide bond in a relatively short period of time. The resulting conjugate is relatively unstable in neutral pH and reverts back to the thiol and maleimide. However, in the presence of alkaline fluid of PDA this reaction undergoes irreversible ring-opening hydrolysis to produce a stable succinamic acid thioether, thereby preventing the elimination of the maleimide-thiol conjugate. Additional network crosslinking may occur through a Michael addition reaction between thiol groups and the C—C bond within the PDA aromatic ring. The crosslinking of GS with PDA may proceed at a slower rate in comparison to the maleimide, and may take several hours or few days to complete, depending on the concentration and pH of the reactants. Furthermore, there are additional interactions that can contribute to the formation of an enhanced network within the hydrogel, thereby improving its mechanical strength. These interactions include but are not limited to covalent bonding between the N—H group of PDA and the C—C within the maleimide ring, as well as hydrogen bonding between the amine groups of GS and the carbonyl groups (C═O) of quinone or maleimide as shown in FIG. 3A.

To analyze chemical interactions during the crosslinking process, comparative FTIR and ¹H NMR spectra of the crosslinked GS-PEM-PDA hydrogel versus its precursor components are shown in FIG. 3B. FTIR spectrum of the GS-PEM-PDA hydrogel showed characteristic peaks of the corresponding precursors including GS, PEM and PDA which are highlighted in FIG. 3B(I). A characteristic peak at 1099 cm⁻¹ represents vibration of —C—O—C— in PEM (labeled as “PEM”), two strong peaks of GS at 1537 cm⁻¹ and 1652 cm⁻¹ (labeled as “GS”) and the shoulder peak of PDA at 1761 cm⁻¹ (labeled as “PDA”) were all present in the spectrum of GS-PEM-PDA hydrogel. To confirm crosslinking of the hydrogel, conversion of maleimide moieties in PEM spectrum was tracked through the distinctive peaks at 1710 cm⁻¹ (corresponding to C═O in maleimide ring) and 695 cm⁻¹ (representing ═C—H in maleimide). Compared to the PEM spectrum, the decrease in intensity of the peak at 695 cm⁻¹ (highlighted in yellow) in the GS-PEM-PDA hydrogel spectrum can be attributed to the Michael reaction crosslinking, which consumes the —C═C— groups of maleimide by reacting with —S—H. Additionally, the C═O peak of maleimide ring at 1710 cm⁻¹ weakened in the GS-PEM-PDA spectrum, suggesting the possibility of hydrolysis and formation of the stable ring-opened succinamic acid thioether.

¹H NMR spectrum of the GS-PEM-PDA hydrogel (FIG. 3B(II)), includes characteristic signals of GS at 3.7 ppm (labeled as “GS”), associating with —CH2 group next to the thiol, and PEM peaks at 3.5 ppm associated to PEG (—O—CH₂—CH₂—O—), and maleimide ring peak at 6.95 ppm (CH₂═CH₂) (labeled as “PEM”) as we can see, this peak in the final gel is reduced due to the covalent bonds with the maleimide part. The ring-opening hydrolysis pathway leads to the corresponding acidic peak at 10.2 ppm. Furthermore, the produced hydrogel has the characteristics of the PDA compound in the form of indole-5,6-quinone. This is exemplified in the aromatic part of the PDA spectrum between 6.5-7.5 ppm, as well as the ═C—H bond of indole structure at 4 ppm (labeled as “PDA”).

The kinetics of hydrogel formation and their mechanical characteristics have an important role in the potential clinical application of injectable hydrogels. To monitor effective crosslinking within a suitable timeframe, dynamic time sweep rheological experiments were conducted. The hydrogels were formed in situ, meaning they were generated on the rheometer stage by combining a mixture of PEM-PDA with GS solution. Since the crosslinking reaction between thiol group and maleimide ring is nucleophilic in nature, the reaction kinetics can be influenced by the pH of the solution. Therefore, the role of pH in gel formation was also investigated using two different sources of PDA with pH=7 and pH=9.5. FIG. 3C(I) and (II) show real-time viscoelastic moduli in response to time, where the crossover point between storage (G′) and loss (G″) moduli were used as an indirect indicator of the gel point. As shown in FIG. 3C(I), gel formation occurred slightly faster under basic conditions, which can be attributed to the favorable environment for thiol groups to act as nucleophiles. Therefore, pH=9.5 was selected for further experiments to investigate the effect of the macromer (precursor) concentration on gelation time and rheological properties. As shown in FIG. 3C(II), increasing the concentration of GS from 10% to 15% wt/v did not significantly change the gel formation time in the absence of PDA. In the presence of PDA, the gel formation time was reduced, potentially due to the formation of additional polymer entanglements and faster reaction between thiol-maleimide at basic pH. However, the impact of PDA concentration was comparatively less pronounced on gel formation time and G′ of the hydrogels. As shown in FIG. 3C(III), regardless of macromer concentrations or pH, the hydrogels formed below 1 min at 37° C. The incorporation of PDA into the network reduced the gel formation time by 20 folds, from ˜40 s for PDA-free hydrogels to ˜2 s for hydrogels containing PDA.

The mechanical assessment of GS-PEM-PDA hydrogels in FIG. 3C(IV) and 3C(V) revealed that increasing GS concentration from 10% wt/v to 15% wt/v in the absence of PDA did not significantly affect the G′ of the hydrogels. Incorporation of alkaline PDA into hydrogels resulted in an elevation of G′ when compared to hydrogels devoid of PDA. In this example, increasing concentration of alkaline PDA improved G′ during the gel formation period (see FIG. 3C(IV)). However, the impact of PDA concentration on G′ after the completion of gel formation at 15 min was observed to be limited (see FIG. 3C(V)). Interestingly, the pH of PDA plays a role on G′ of the hydrogels both at gel formation time and after the completion of gel formation at 15 min. It was found that, in this example, introducing PDA at pH=9.5 improved G′ by >350 fold at gel formation time as compared with PDA-free hydrogels. The observed reduction in gel formation time and increased mechanical strength of hydrogels containing alkaline PDA, plays a role in preventing dissolution or removal of the material from its injected site during the gel formation time. Moreover, the increased mechanical strength of the hydrogels containing alkaline PDA at least partially mitigates potential risks of leakage or displacement of hydrogels from the fistula tract over time, and may result in better healing efficacy in clinical applications.

### Mechanical and Physical Properties of Hydrogels

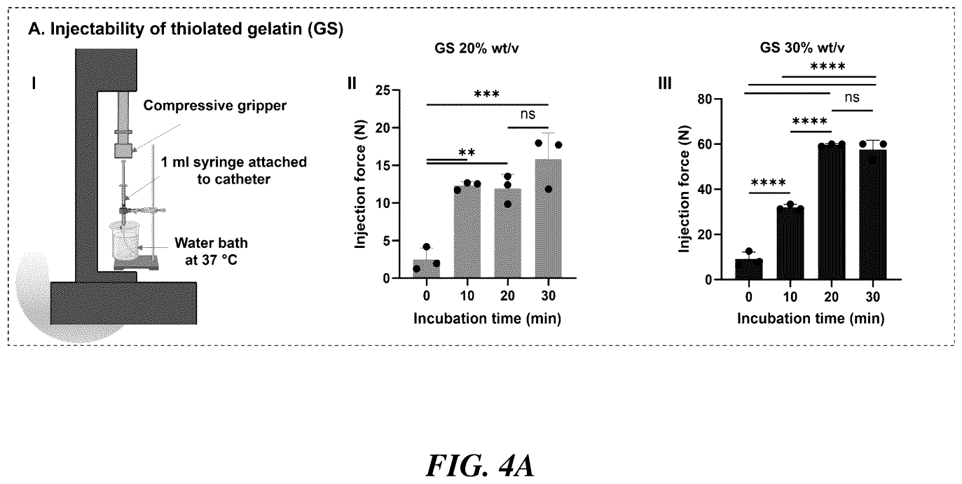

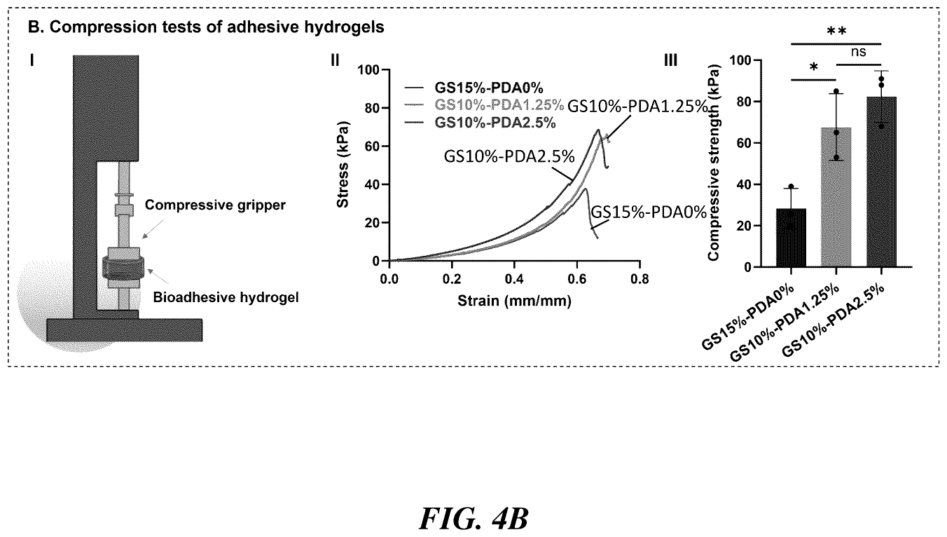

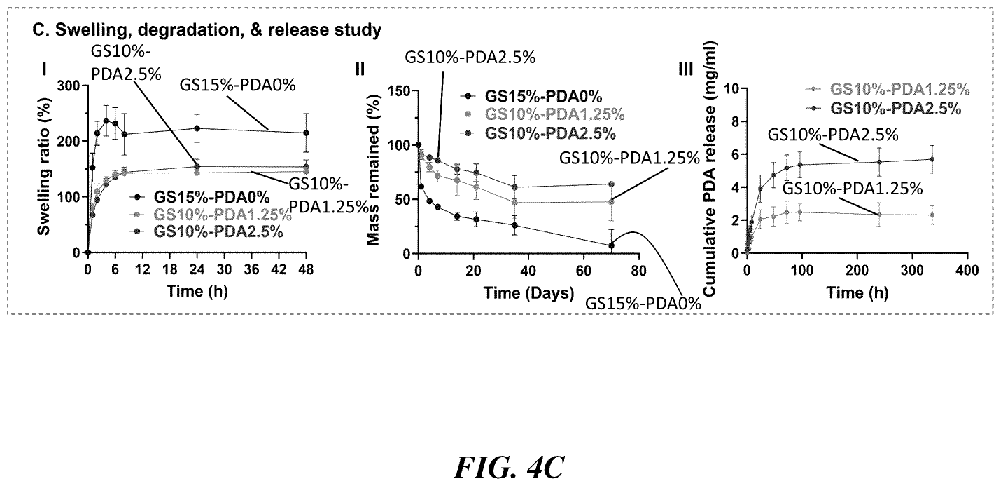

Given the fact that viscosity of GS solution at room temperature increases over time, potentially hindering ease of injection and leading to catheter blockage, it is important to quantify the force needed for injecting the GS solution through the applicator across different time points. For this purpose, the GS solution, at a temperature of 37° C., was transferred into a 1 ml syringe connected to a 4F catheter (11 cm in length) and placed in the Instron mechanical testing equipment (FIG. 4A(I)). As the catheter remains within the body throughout the injection period, the catheter was submerged in a 37° C. bath to simulate physiological temperature, whereas the syringe filled with GS was kept at room temperature (˜ 25° C.) during the experimental period. The injectability of various GS concentrations (20% wt/v and 30% wt/v) was assessed over 30 min. As depicted in FIG. 4A(II), the injection force for GS 20% wt/v was ˜15 N after 30 min. The GS at a concentration of 20% wt/v was manually injectable within the acceptable force range (<40 N), with no observed catheter blockage over the 30 min period. Nevertheless, increasing GS concentration to 30% wt/v seemed to result in an elevated injection force attributed to accelerated solidification (FIG. 4A(III)). Consequently, the material became non-injectable through the catheter after 20 min. Without wishing to be bound by any particular theory, the integration of PDA in the hydrogel may have expedited the gel formation and elevated the mechanical properties by ˜350 times, in this exampled, which effectively offset the challenge of using higher concentrations of GS (30% wt/v) to increase the mechanical properties.

An effective bioadhesive may possess adequate mechanical strength to maintain its original shape when compressed by the injured tissue. To assess the stability of our hydrogels under physiological load-bearing conditions, hydrated devices were subjected to uniaxial compressive tests, as illustrated in FIG. 4B(I). Our investigation focused on exploring the impact of different PDA concentrations (0%, 1.25%, and 2.5% w/v) on the compressive strength of the hydrogels. The stress-strain curves depicted in FIG. 4B(II) highlight ˜60%-70% elasticity for all the hydrogels. With an increase in PDA concentration from 0% to 1.25% wt/v, and 2.5% wt/v, an improvement in compressive strength was observed, in this example, rising from 28.2 kPa to 67.6 kPa, and 82.3, respectively (FIG. 4B(III)).

Physical assessments, including the determination of hydrogel swelling rates, are parameters that allow the hydrogel to expand and conform to the distinct morphology of the fistula. This process facilitates the establishment of a secure seal and occlusion of the cavity. The swelling behavior of GS-PEM-PDA bioadhesives at varying concentrations of PDA was investigated over a 48-h time period. The graph in FIG. 4C(I) illustrates that equilibrium swelling was achieved within the initial 8 h for all hydrogels, after which the swelling stabilized. In this example. hydrogels lacking PDA content exhibited a relatively high swelling ratio of ˜220%, indicative of improved hydrophilicity. Upon the introduction of PDA to the hydrogels, the swelling ratio decreased to ˜150% which may be attributed to the robust crosslinking mechanism facilitated by PDA, leading to a diminished solvent absorption capacity.

Biodegradation helps the hydrogel break down gradually in sync with the body's healing process, promoting effective closure of the fistula over time. FIG. 4C(II) displays the degradation rate of GS-PEM-PDA bioadhesives over the course of 10 weeks in collagenase (2.5 U/ml) at 37° C. By day 70, the remaining mass of bioadhesives reduced to ˜7.5%, ˜48% and ˜66% for PDA 0%, 1.25% and 2.5% wt/v, respectively. These findings suggest that hydrogels with higher PDA concentrations can exhibit relatively higher durability in situ.

Since antibacterial and healing properties of our hydrogels are associated with PDA presence in the fistula microenvironment, evaluating the kinetics of PDA release is crucial for determining sustained antibacterial and healing effects. FIG. 4C(III) exhibits cumulative PDA release from GS-PEM-PDA hydrogels at different initial PDA concentrations over a two-week period in DPBS at 37° C. The hydrogels begin to release PDA within 1 h with a continual increase observed up to 96 h, followed by sustained release persisting up to 336 h. Irrespective of the initial concentration of PDA in the hydrogels, a total of 20% PDA was released from the hydrogels. Notably, doubling the initial PDA concentration in the hydrogels from 1.25% to 2.5% resulted in a twofold increase in the total amount of PDA released, corresponding to ˜2.3 mg and ˜5.7 mg of released PDA from 1 ml of the hydrogel, respectively.

### In Vitro Adhesion and Ex Vivo Occlusive Properties of Hydrogels

The adhesive property of occlusive hydrogels to the tissue is crucial to ensure proper sealing and non-displacement within the fistula tract. and it must be tested to determine their sealing efficacy before being applied in vivo. The schematic in FIG. 5A(I) depicts the procedure for in vitro wound closure test. An artificial wound was made by blade on porcine skin and the bioadhesive hydrogels were applied on the surface of the wound and allowed to crosslink for 15 min to close the wound. The adhesive strength was then evaluated by tensile test using Instron mechanical testing machine until the biadhesive hydrogel failed. In the wound closure test, the hydrogels containing PDA showed significantly higher adhesive strength compared to the PDA-free hydrogel and commercial fibrin-based glue, Tisseel. The shear strength of the bioadhesives was evaluated at room temperature using porcine skin, following the modified ASTM F2255-05 standard for tissue adhesives as schematically shown in FIG. 5A(II). In the shear strength test, hydrogels containing PDA showed higher shear strength than PDA-free hydrogels. Incorporation of the PDA into the hydrogels increased the shear strength by ˜3-fold higher than the commercial glue, Tisseel, perhaps due to dual in situ crosslinking and physical interactions of PDA with functional groups of tissue.

To assess the hydrogels efficacy to seal a fistula tract, an ex vivo bench test was conducted. As shown in FIG. 5B(I), the setup involved a pulsating pump driving a warm DPBS solution through silicone tubes connected to a fistula model (with a diameter of 7 mm and a length of 5 cm) created within a pig belly fat tissue. The model was then filled with hydrogels containing varying concentrations of PDA or commercial glue, Tisseel, using a dual-lumen 4 F catheter (11 cm in length). The pressure of the system was continuously monitored using a manometer (PDA 100L, PCE) attached to a computer. As the DPBS circulated through the system at different flow rates (30, 60, 90, and 120 ml/min), the pressure gradually rose, until the hydrogel broke or displaced. A minimum pressure of 300 mmHg, simulating the pressure induced by patient movement, was set as a threshold to evaluate the success rate of the hydrogels. Based on the aforementioned experimental setup, the hydrogel can be effective at occluding fistula and/or other body cavities, as it demonstrated resilience against physiological pressures and movements at the highest flow rate of 120 ml/min (mimicking arterial blood pressure).

As depicted in FIG. 5B(II) at a low flow rate of 30 ml/min, all the hydrogels could withstand a minimum pressure of 300 mmHg, comparable to Tisseel. However, at higher flow rate of 120 ml/min, only the hydrogels containing PDA (1.25% and 2.5% wt/v) could reach to minimum 300 mmHg (FIG. 5B(III)), with 100% success rate in occluding the fistula tract (FIG. 5B(IV)).

### Assessment of Hemocompatibility and Cytocompatibility of the Hydrogels

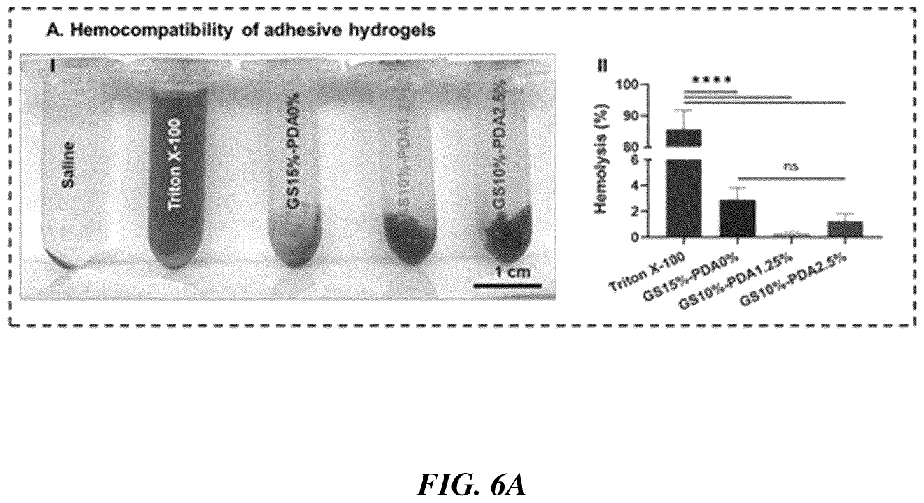

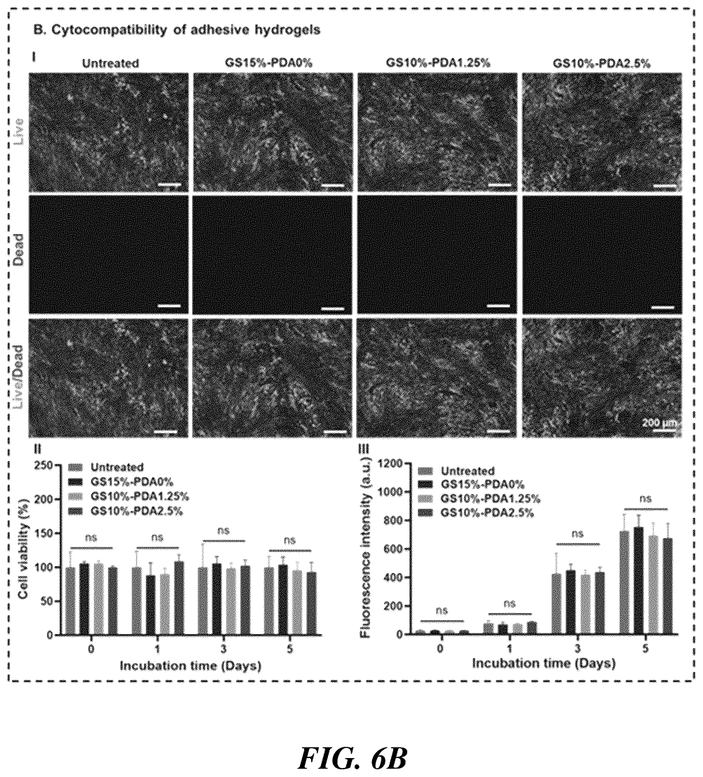

In the development of bioadhesives, it is important to assess hemocompatibility to evaluate whether the materials damage the blood cells. For this purpose, a standard hemolysis test was performed by exposing crosslinked hydrogels to diluted sodium citrate-treated whole human blood for 2 h at physiological temperature. The presence of a toxic effect was assessed visually by observing the appearance of a red-colored supernatant after centrifugation, indicating the release of hemoglobin from ruptured red blood cells (RBCs). As depicted in FIG. 6A(I), no significant color change was observed in the supernatant following incubation of the hydrogels with blood across all concentrations of PDA. After incubation with hydrogels, intact RBCs were efficiently sedimented through centrifugation, similar to the saline negative control. However, the positive control, Triton X-100, displayed a reddish supernatant, indicative of its hemolytic effect. The disruption of RBCs was further assessed through colorimetric analysis, with the calculated percent hemolysis induced by the hydrogels presented in FIG. 6A(II). The hemolysis level of the hydrogels at all tested PDA concentrations remained below the acceptable limit (≤5%). These findings suggest that the GS-PEM-PDA hydrogels can be potentially used for their therapeutic effects without causing disruption to RBCs.

In addition to hemocompatibility, bioadhesives should support biological activity and promote wound closure without hindering cell proliferation. Therefore, cell viability and proliferation was assessed in the presence of GS-PEM-PDA hydrogels using live/dead viability assays and PrestoBlue metabolic activity assays. FIG. 6B(I) presents fluorescent images of live and dead human dermal fibroblast (HDF) after incubating with hydrogels for 5 days. The untreated cells were allowed to grow in the cell culture plate without subjecting to hydrogel. The majority of the hydrogel-treated cells appeared green indicating high viability, and only a few cells stained red representing dead cells. FIG. 6B(II) illustrates the quantitative percentage of cell viability. Notably, the incorporation of PDA did not negatively impact on the cell viability and all the hydrogels maintained cell viability of >90% with non-significant differences from the untreated control. FIG. 6B(III) demonstrates a consistent increase in fluorescence intensity of PrestoBlue reagent in the presence of hydrogels over a 5-day period, indicating efficient metabolic activity and cell proliferation across all the concentrations of PDA.

### In vitro anti-infective property and wound healing effect of the hydrogels

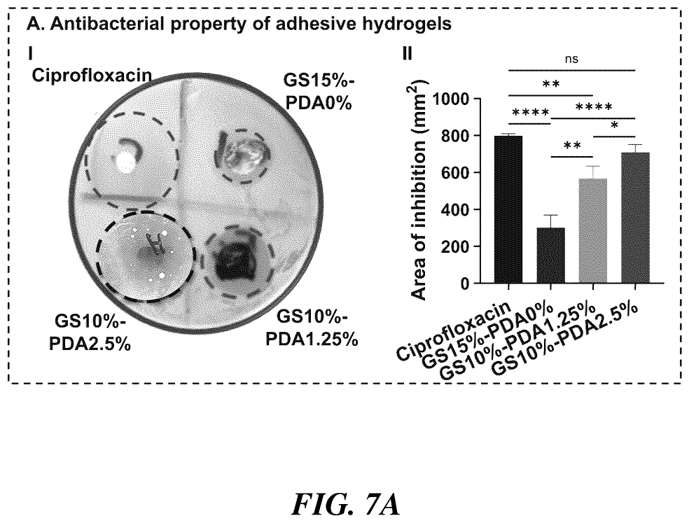

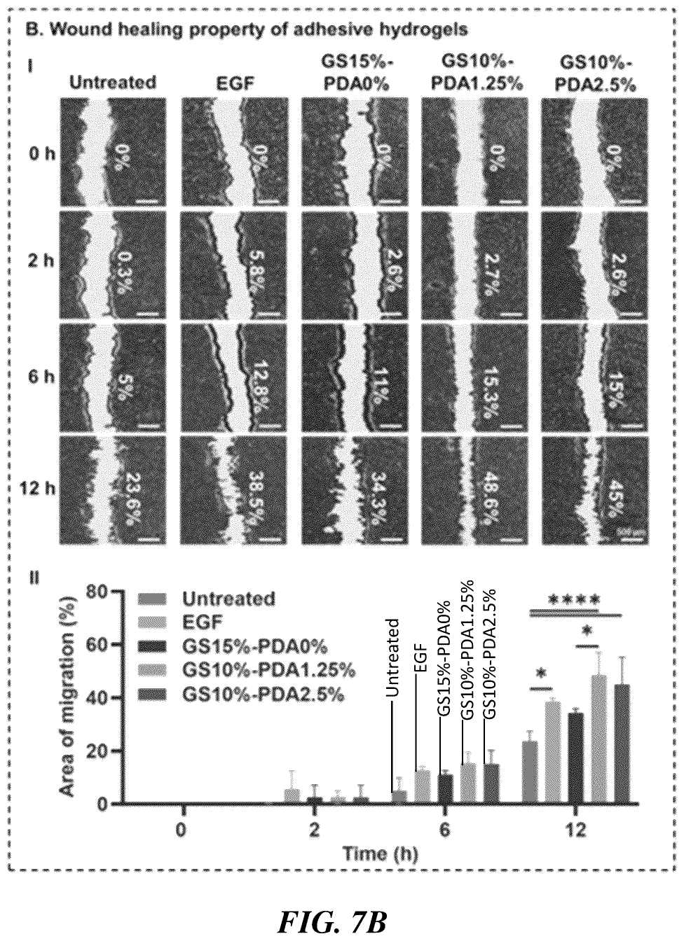

To assess the anti-infective capability of the bioadhesive hydrogels, a source of bacterial infection in anal fistula, namely Escherichia coli (E. coli, Gram-negative), was used as a model bacterium. The hydrogels at different concentrations of PDA were sterilized under UV light for ˜1 hour and were then soaked in sterile DPBS for 1 h to hydrate. Ciprofloxacin (8 mg/ml) served as the positive control. In the zone of inhibition test (FIG. 7A(I)), the anti-infective effect was determined by the presence of clear bacteria-free zones surrounding the hydrogels and compared with the inhibition zone of Ciprofloxacin. FIG. 7A(II) shows that the area of inhibition in E. coli culture did not differ significantly between Ciprofloxacin and hydrogels containing 2.5% wt/v of PDA. Overall, the PDA-free hydrogels demonstrated ˜37% antibacterial effect, whereas incorporation of 1.25% and 2.5% wt/v PDA improved the anti-bacterial effect to 71% and 89%, respectively. Although the precise anti-infective mechanism of our hydrogels is unclear, the interaction between the released PDA with the bacterial cell wall could be one of the possible mechanisms.

FIG. 7B(I) displays an assessment of in vitro wound healing over a 12-hour period. An artificial wound was created by cell scratching on well plates. The cells left for natural healing served as the untreated control, the cells grown in the presence of the hydrogels elute containing a 7-day released PDA were considered as experimental group, and the cells grown in the presence of epidermal growth factor (EPG) served as positive control. ImageJ software (Version 1.52e, National Institute of Health, USA) was utilized to calculate the total wound surface area in each well at different time points (0, 2, 6, and 12 h). The results indicated that bioadhesives containing PDA significantly enhances wound closure as compared with untreated control, similar to the EPG. Specifically, within 12 hours, about half of the wound was closed with GS-PEM-PD containing 1.25% and 2.5% wt/v PDA, whereas with PDA-free hydrogels and untreated control only, wound closure was ˜23.6% and ˜34.3%, respectively (FIG. 7B(II)). These results highlight the efficacy of our bioadhesives for not only sealing the wound but also promote wound healing.

### Conclusion

In conclusion, this example presented a novel approach to treat fistula using regenerative and occlusive bioadhesive hydrogels. The inclusion of PDA in this injectable hydrogel, along polyethylene glycol maleimide and thiolated gelatin, conferred a multifaceted functionality, serving as a bioadhesive, antibacterial agent, and tissue regenerator. The GS-PEM-PDA hydrogels transit relatively quickly into a gel state, exhibiting adhesive properties to tissue that are three times more potent than the commercial fibrin-based glue. Moreover, the integration of PDA resulted in improved mechanical and occlusive properties, allowing the hydrogel effectively block tubular structures under physiological pressure.

GS-PEM-PDA hydrogels, containing 1.25% wt/v and 2.5% wt/v of PDA with their elevated crosslinking density, demonstrated a slower degradation rate compared to PDA-free gels. This characteristic suggests the material's relative stability within the body during the required treatment time.

The hydrogels were cytocompatible and hemocompatible, showing the potential for their safe application in further in vivo studies. The PDA-infused hydrogels demonstrated an antibacterial efficacy of over 70% and about twofold increase in wound healing, in vitro.

In sum, our results suggested that these injectable hydrogels have exciting potential for fistula tract closure. All the gels containing PDA withstood pressures of up to 300 mmHg which simulated the pressure induced by patient movement. Accordingly, this bioengineered hydrogel may potentially reduce the failure rate of fistula treatment by successfully occlude and heal abnormal cavities, substantially improving the quality of life of the patients.

### EXAMPLE 2

The following example generally relates to the formation of a cross-linked polymer network comprising a contrast agent and a peptide to accelerate wound healing.

### Preparation of GS-PEM-PDA bioadhesive hydrogels containing contrast agent and BPC-157

Preparation of fluid 1: thiolated gelatin (GS) (20% wt/v) was added to Milli-Q water, and a Omnipaque (300 mg/ml) contrast agent (50% v/v) and BPC-157 (0.1% wt/v in Milli-Q water) were mixed with GS, followed by incubation at 37° C. until complete dissolution.

Preparation of fluid 2: A 1:1 mixture of aqueous PDA (0%, 2.5%, or 5% wt/v, pH=9.5), and PEM (20% wt/v in DPBS, pH˜7.4) was prepared.

The hydrogel was obtained by combination of equal volume of fluid 1 with fluid 2 to allow gel formation.

### Result and Discussion

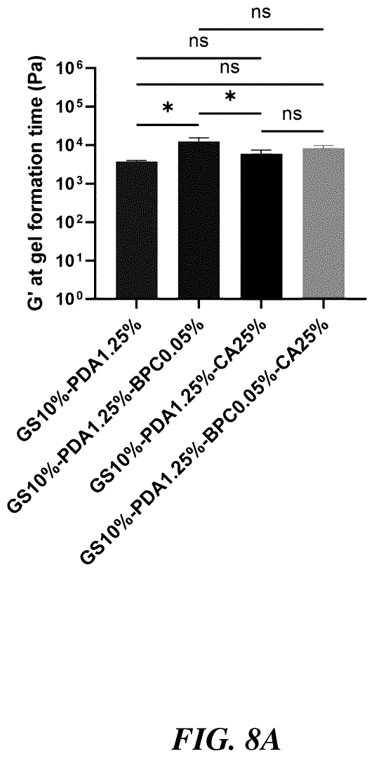

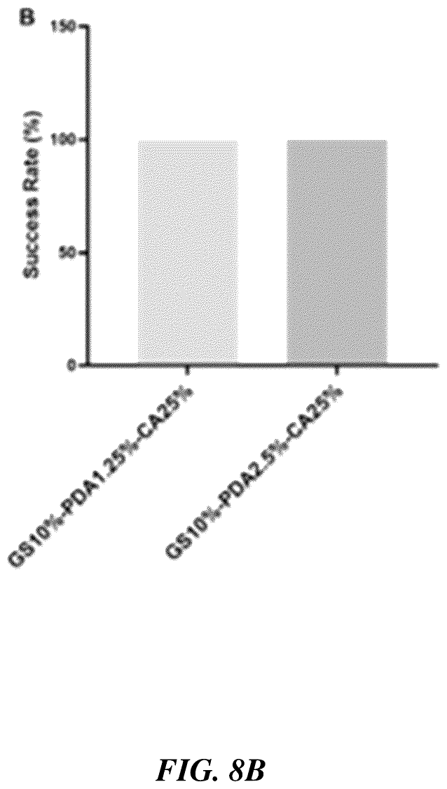

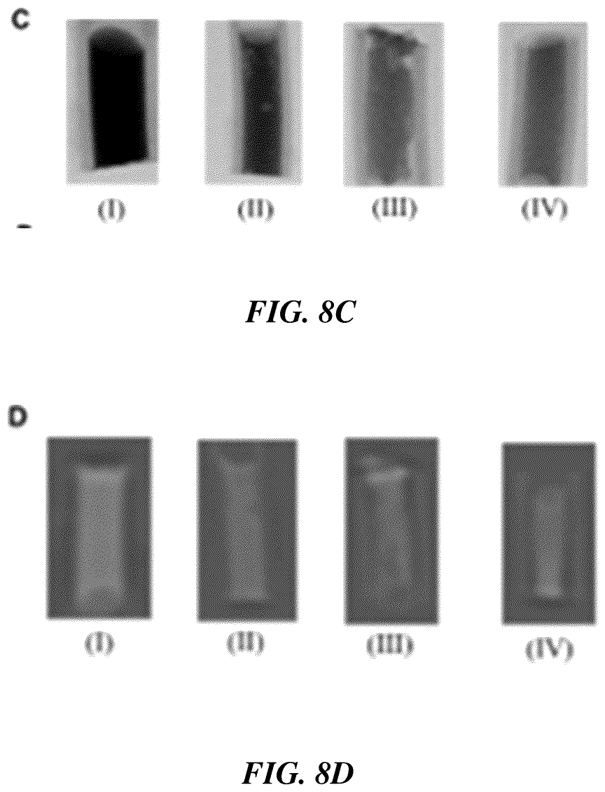

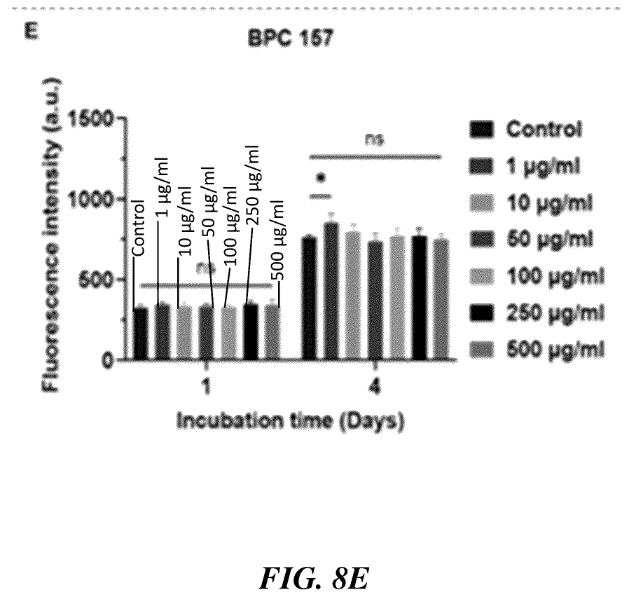

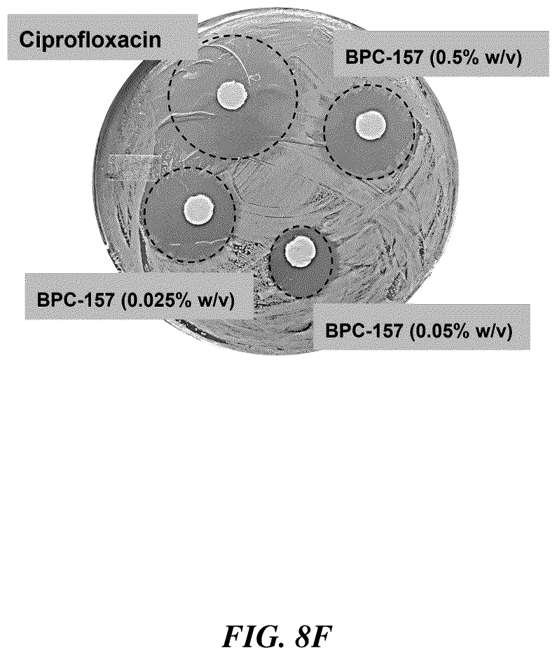

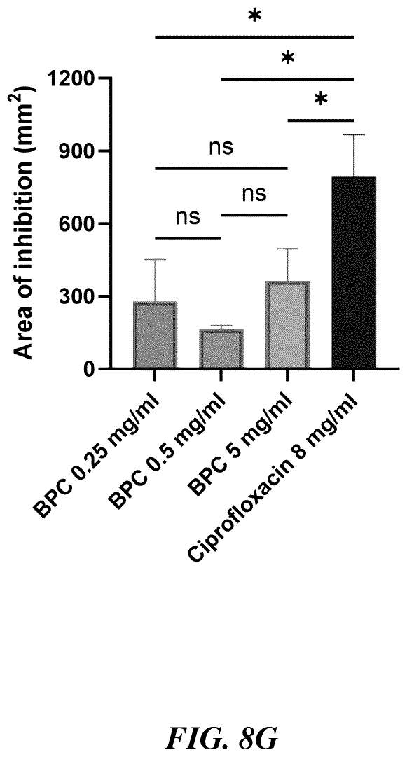

The mechanical assessment of hydrogels at gel formation time, as illustrated in FIG. 8A, demonstrates the influence of the contrast agent (CA, Omnipaque 300 mg/ml) and anti-inflammatory drug (BPC-157) on the storage moduli (G′) of the hydrogels. In this Example, the addition of the BPC-157 (final concentration of 0.05% w/v) to the GS10%-PEM10%-PDA1.25% hydrogels led to an enhancement in G′ as compared to the drug-free GS10%-PEM10%-PDA1.25%. Conversely, the incorporation of CA (final concentration of 25% v/v) to the GS10%-PEM10%-PDA1.25% hydrogels, resulted in a significant decrease in the G′ of the drug-and CA-free hydrogels. However, simultaneous incorporation of the drug and CA did not negatively impact on mechanical properties of the GS10%-PEM10%-PDA1.25% hydrogels.

Injection of the hydrogels containing CA (final concentration of 25% v/v) to the fistula model (diameter=5 mm, length=1 cm) could occlude the tract with 100% success rate at a flow rate of 120 ml/min and pressure of 300 mmHg, regardless of the PDA concentration in the hydrogels. This result emphasizes the potential of the radiopaque hydrogels to successfully occlude fistula in vivo.

Radiopacity evaluation of different hydrogels versus stand-alone CA was evaluated under fluoroscopy (FIG. 8C) and CT scan imaging (FIG. 8D). As could be seen in these figures, sample II, containing mixture of GS20% and CA (50% v/v) showed radiopacity with the intensity similar to the sample I, containing stand-alone CA (100% v/v). Interestingly, the crosslinked hydrogels, namely GS10%-PEM10%-PDA2.5%-CA25% (sample III), and GS10%-PEM10%-PDA1.25%-CA25% (sample IV) were also radiopaque. However, owing to the lower concentration of the CA in the crosslinked hydrogels, the intensity of the images was low in sample III and IV as compared to sample I and II.

FIG. 8E demonstrates a consistent increase in fluorescence intensity of PrestoBlue reagent in the presence of BPC-157 over a 4-day period, indicating efficient metabolic activity and cell proliferation across all the concentrations of the drug tested (0-500 μg/ml). No cytotoxicity effect was observed after incubation of the drugs with HDF cell for 4 days.

In the zone of inhibition test (FIG. 8F), the anti-infective effect of different concentrations of BPC-157 against E. coli was determined by the presence of clear bacteria-free zones surrounding the hydrogels and compared with the inhibition zone of Ciprofloxacin (8 mg/ml). FIG. 8G shows the area of inhibition in E. coli culture was independent of BPC-157 concentrations. BPC-157 showed ˜45% antibacterial effect as compared to the Ciprofloxacin (8 mg/ml).

In summary, this example shows the feasibility of the hydrogel to carry different therapeutics and radiopaque agents without affecting on their mechanical and occlusion properties. BPC-157 showed cytocompatibility and antibacterial properties which would be desirable features for fistula treatment.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

In the claims, as well as in the specification above, all transitional phrases such as “comprising,” “including,” “carrying,” “having,” “containing,” “involving,” “holding,” “incorporating”, “integrating” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
polydopamine;
a maleimide-functionalized cross-linker; and
a thiolated-polymer.

2. The article of claim 1, further comprising norepinephrine, adrenaline, catecholamine, 4-(2-aminoethyl)-2-methoxyphenol hydrochloride, 5-(2-aminoethyl)pyrogallol hydrochloride, 4-methylpyrocatechol, 3-methylpyrocatechol, 3-fluoropyrocatechol, 3,4-dihydroxybenzonitrile, o-ethoxyphenol, 2-methoxy-4-methylphenol, 4-ethylpyrocatechol, 4-(aminomethyl)pyrocatechol hydrobromide, 3-methoxypyrocatechol, 3,4-dihydroxybenzyl alcohol, o-methoxybenzenethiol, 4-chloropyrocatechol, 4-tert-butylpyrocatechol, o-diethoxybenzene, 4-allyl-1,2-dimethoxybenzene, 3,4-dimethoxy-6-methylpyrocatechol, 4-phenylpyrocatechol, 2-chloro-3′,4′-dihydroxyacetophenone, sodium 2-methoxy-5-nitrophenolate, o-phenylene diacetate, o-(benzyloxy)phenol, (+-)-4-(1-hydroxy-2-(isopropylamino) ethyl)-1,2-benzenediol hydrochloride, 3,4-dihydroxybenzophenone, 3,5-di-tert-butylpyrocatechol, potassium 3,4-dihydroxybenzenesulfonate, tetrachloropyrocatechol, pyrocatechol dipentyl ether, 4-(p-nitrophenylazo) catechol, (+)-catechin, (−)-epicatechin, (+-)-p-(2-(3-(p-hydroxyphenyl)-1-methylpropylamino)ethyl)pyrocatechol hydrochloride, 4,4′-(2,3-dimethyltetramethylene)dipyrocatechol, 4-nitrocatechol sulfate dipotassium salt, disodium 4,5-dihydroxy-1,3-benzenedisulfonate (Tiron), pyrocatechol violet, caffeic acid, polycaffeic acid, tanic acid, 1,2,4-benzenetriol, pyrogallol, coniferaldehyde, 2,4-dinitrophenol, 4-nitrocatechol, and 4-methylcatechol, and/or humic acid.

3. The article of claims 1, further comprising an anti-inflammatory agents, wound healing accelerator agents, and/or growth factors.

4. The article of claim 3, wherein the anti-inflammatory and/or wound healing accelerator agents comprise polydopamine, pentadecapeptide BPC 157, cyclo-gly-pro (cyclic peptide), and/or prostaglandin.

5. The article of claim 3, wherein the growth factors comprise epidermal growth factor, transforming growth factor, vascular endothelial growth factor, fibroblast growth factor, platelet derived growth factor, and/or keratinocyte growth factor.

6-8. (canceled)

9. The article of claim 1, wherein the maleimide-functional crosslinker comprises at least 1 arm and molecular weight at least 1 kDa.

10. The article of claims 1, wherein the polydopamine is present in the article in an amount greater than or equal to 0.01% w/v and less than or equal to 5% w/v.

11. The article of claim 1, wherein the polydopamine comprises a plurality of particles.

12. (canceled)

13. The article of claim 1, wherein the maleimide-functional crosslinker comprises poly(ethylene glycol) maleimide, maleimide, N-(2-Aminopropyl) maleimide, N-ethylmaleimide, N-phenylmaleimide, N-methylmaleimide, n-cyclohexylmaleimide, N-benzylmaleimide, N-(p-tolyl) maleimide, N-(p-tolyl) maleimide, N-(p-aminophenyl) maleimide, 1,2,3,4-cyclobutanetetracarboxdiimide, N-(p-methoxyphenyl) maleimide, N-(o-chlorophenyl) maleimide, n-cyclohexyl maleimide, N-(p-nitrophenyl) maleimide, 3,4-dibromomaleimide, N-bromomethyl-2,3-dichloromaleimide, fluoroimide, N,N′-(p-phenylene) dimaleimide, N,N′-o-phenylenedimaleimide, N,N′-m-phenylenedimaleimide, 1,6-bismalemidohexane, N-(2,4,6-trichlorophenyl) maleimide, N-(p-(phenylazo) phenyl) maleimide, N-(4-(2-benzimidazolyl) phenyl) maleimide, N-(9-acridinyl) maleimide, N-(3-fluoranthenyl) maleimide, 7-dimethylamino-3-maleimido-4-methylcoumarin, N-(4-dimethylamino-3,5-dinitrophenyl) maleimide, 2-benzylamino-N-(p-methoxyphenyl) maleimide, N,N′-(methylenedi-p-phenylene) dimaleimide, 1,1′-(methylenebis(2-ethyl-6-methyl-4,1-phenylene)) dimaleimide, N-(2-Hydroxyethyl) maleimide, N-Maleoyl-β-alanine, Alkyne-PEG4-maleimide, Dibenzocyclooctyne-maleimide, Methoxypolyethylene glycol maleimide, N-Biotinoyl-N′-(6-maleimidohexanoyl) hydrazide, Acetylene-PEG4-maleimide, DBCO-PEG4-maleimide, 5-Maleimido-fluorescein, Maleimide-PEG-OH, 2-Maleimidoethylamine trifluoroacetate salt, N-(2-Hydroxyethyl) maleimide, 1,1′-(Methylenedi-4,1-phenylene) bismaleimide, 1,1′-(Methylenedi-4,1-phenylene) bismaleimide, 1,1′-(hexane-1,6-diyl) bis(1h-pyrrole-2,5-dione), and/or maleic anhydride.

14. The article of claim 13, wherein poly(ethylene glycol) maleimide is present in the article in an amount greater than or equal to 0.01% w/v and less than or equal to 20% w/v.

15. The article of claim 1, wherein the thiolated-polymer comprises thiolated-hyaluronic acid, thiolated-chitosan, thiolated-collagen, thiolated-gelatin, thiolated-elastin, thiolated-elastin-like polypeptides, thiolated-cysteine residues, thiolated-alginate, thiolated-polygalacturonic acid, thiolated-β-Cyclodextrin, thiolated-poly(ethylene glycol), thiolated-dextran, thiolated-heparin, and/or thiolated-glycogen.

16-23. (canceled)

24. An article, comprising:
an adhesion promoter selected from the group consisting of, indole-5,6-quinone, eumelanin, melanin, pheomelanin, 5,6-dihydroxyindole, tyrosine, L-dopaquinone, L-leucodopachrome, serotonin, and L-dopachrome, caffeic acid, polycaffeic acid and tanic acid, a multi-functional crosslinker; and a thiolated-polymer.

25-39. (canceled)

40. The article of claim 24, wherein the article has a compressive strength greater than or equal to 15 kPa.

41. The article of claim 24, wherein the article has an adhesive strength greater than or equal to 10 kPa.

42. The article of claim 24, wherein the article swells less than 225% when in contact with DPBS is for 24 hours at 37 degrees Celsius.

43. The article of claim 24, wherein the storage modulus of the article is greater than or equal to 10 Pa at the onset of gelation.

44-46. (canceled)

47. A method, comprising:
administering a mixture into a body cavity in a subject, wherein the mixture comprises a first fluid and a second fluid, the first fluid comprising polydopamine and a maleimide-functionalized crosslinker, and the second fluid comprising a thiolated-polymer.

48. The method of claim 47, wherein upon administering the mixture, the first fluid comes into physical contact with the second fluid to form a cross-linked polymer network.

49. The method of claim 47, wherein the cross-linked polymer network adheres to a portion of the body cavity.

50-80. (canceled)

81. The method of claim 47, wherein the cross-linked polymer network can withstand a pressure greater than or equal to 10 mmHg at flow rate of 120 ml/min.

82-85. (canceled)
